(19) United States
(12) Patent Application Publication
(10) Pub. No.: US 2011/0076712 A1
(43) Pub. Date: Mar. 31, 2011

(54) LUBRICIOUS MICROFLUDIC FLOW PATH SYSTEM

(75) Inventors: Thomas Boyd Gilligan, College Station, TX (US); Kenneth Michael Evans, College Station, TX (US)

(73) Assignee: XY, LLC., Navasota, TX (US)

(21) Appl. No.: 12/992,303

(22) PCT Filed: Jun. 12, 2009
(86) PCT No.: PCT/US09/03527

§ 371 Date: Nov. 12, 2010

Related U.S. Application Data
(60) Provisional application No. 61/132,001, filed on Jun. 13, 2008.

(51) Int. Cl. C12Q 1/02; C12M 1/34

(52) U.S. Cl. 435/29; 435/288.7

(57) ABSTRACT

A flow cytometer having a nozzle assembly which provides a lubricious flow path through which a plurality of particles pass to be entrained in a flow of liquid to provide a faster method for sorting with increased cell quality.

### I. BACKGROUND

Generally, a lubricious microfluidic flowpath for a particle analysis device. Specifically, a flow cytometer having a nozzle assembly which provides a lubricious flow path through which a plurality of particles pass to be entrained in a flow of liquid.

A number of conventional instruments are available for the separation of a plurality of particles based on differentiable characteristics into two or more populations. Typically, it is desirable to maximize the speed at which the separation based upon the differentiable characteristic(s) occurs, the purity of the isolated population(s) separated based upon the differentiable characteristic(s), and the yield of each of the separated population(s) in number of particles in relation to the total number particles introduced into the separation process.

Typically, it is possible to increase any one of speed, purity or yield; however, this is generally achieved by decreasing the other two. The decrease is most dramatic as the differentiable characteristics of any two types or kinds of particles approaches zero under given set of analysis conditions. As one non-limiting example, the amount of deoxyribonucleic acid (“DNA”) differs between X chromosome bearing sperm cells and Y chromosome bearing sperm cells. This difference in the amount of DNA between X chromosome bearing sperm cells and Y chromosome bearing sperm cells varies between mammalian species and certain non-mammalian species. The difference in the amount of DNA between X chromosome bearing sperm cells and Y chromosome bearing sperm cells in cattle is significantly greater than the difference in the amount of DNA between X chromosome bearing sperm cells and Y chromosome bearing sperm cells of certain primates. Accordingly, the separation of X chromosome bearing sperm cells and Y chromosome bearing sperm cells of cattle into isolated populations can be substantially less difficult than the separation of X chromosome bearing sperm cells and Y chromosome bearing sperm cells of certain primates into isolated populations each based on the difference in the amount of DNA.

With respect to the non-limiting context of separating sperm cells by flow cytometry based upon the occurrence of an X chromosome or a Y chromosome in a given one of a plurality of sperm cells, “purity” means the percentage of sperm cells in an isolated population of sperm cells having the desired chromosome. For example, a population of sperm cells which contains 90% percent X-chromosome bearing sperm cells and 10% Y-chromosome bearing sperm cells can be said to have a purity of 90%. Accordingly, the purity of an unsorted population of sperm cells can be said to be 50%.

One approach to generate a higher purity of separated particle populations can be to reduce the variation in the analysis between particles. For example, with respect to the analysis of sperm cells by flow cytometry, although the amount of chromosomal DNA contained in each one of a plurality of X-chromosome bearing sperm cells is exactly the same number of nucleotides, upon interrogation of each X-chromosome bearing sperm cell during analysis by flow cytometry the amount of emitted light from each X-chromosome bearing sperm cell (as further described below) can vary due to variations in the rotational position, variations in the axial location within the fluid column (also referred to as centricity), variations in the incidence of the illuminating light on the fluid column, variations in the amount of fluorochrome dye bound to the chromosomal material, variation in the amount of quenching dye having passed the sperm cell membrane, variations in the intensity of the laser light, differences in the amount of mitochondrial DNA, and differences in the amount of NADH/NAD⁺, or the like. Any means or method which reduces variation of an analysis between particles, and specifically the above-mentioned variations relating to the analysis of sperm cells, can correspondingly increase the resolving power of the analysis which can result in the collection of populations of particles with greater purity at equivalent yield and speed.

Specific to the separation of sperm cells in the flow cytometry context, another parameter related to purity can be co-incidence of more than one sperm cell occurring in a droplet breaking off from the fluid stream exiting the nozzle of the flow cytometer. Co-incidence is a mathematical relationship between the position of any one sperm cell in the fluid stream of analysis compared to the positions of the sperm cells just prior to and just following that one sperm cell in the stream of analysis combined with the rate at which droplets are formed upon exit of the fluid stream from the nozzle of the flow cytometer.

To determine the co-incidence value (statistical chance of particles being entrained within the same drop), the rate of drop formation, and the drop-delay calibration are required. The rate of drop formation is typically controlled by a regulator that adjusts the oscillation rate of a piezoelectric crystal (for example 60,000 Hz) which provides motive force to the nozzle, causing the fluid stream to break into droplets at the same rate of oscillation. The drop delay is determined by the rate of fluid flow and the drop formation rate and is commonly expressed in fractional drops to within 1/16 of a drop formation period or about 10-6 Hz (for example 22-3/16).

Although co-incidence is a “positive qualifier” (meaning it describes the positive attribute of two particles having a high probability of coinciding in the same drop), for the purposes of sorting particles, co-incidence is typically an unwanted event, as it typically contributes to the collection of contaminating particles correspondingly reducing purity of the collected populations of particles. Accordingly, drops with a highly probable co-incidence are usually not collected. The co-incidence rate is value expressing the number of such co-incidences occurring within a specified period of time (for example 1000 co-incidence events per second). The rates of coincidence are easily reduced by reducing the rate at which particles are entrained within the analysis stream, without varying the volume of fluid flow or rate of drop formation.

A substantial problem with increasing purity by increasing centricity of a particle in a fluid stream typically by reduction in the cross sectional area of the fluid stream can be a reduction in the flow rate of the fluid stream at a given pressure which correspondingly reduces yield of the analysis. Also, reducing the rate at which particles are entrained within the analysis stream to reduce co-incidence further reduces the yield of the analysis. Accordingly, particle analysis by conventional microfluidic devices, and specifically in the context of flow cytometry, can be substantially limited by the inability to achieve higher flow rates through lesser internal diameter microfluidic conduits.

For the purpose of the instant invention, “yield” is the percentage of desired particle isolated or sorted away from the total number of particles entering the analysis. As a non-limiting example, if a mixed population of X-chromosome bearing sperm and Y-chromosome bearing sperm are entering the analysis at 40,000 incoming sperm per second, and 4000 X-chromosome bearing sperm are sorted, then the yield is said to be 10%.

If the particle population being sorted is an abundant material with low cost, then low yields are acceptable, while if the material is a rare or valuable material, high yields are desired. As a non-limiting example, 20 billion fresh bovine sperm from a proven bull with high genetic value may be used by those skilled in the art to produce about 1000 units of cryopreserved bull semen that has not been sex sorted, assuming 20 million sperm are included in each straw. If enough sorters are utilized to sex sort the entire amount of 20 billion fresh sperm, with a yield of 10%, only 100 units of cryopreserved sexed semen can be produced. Accordingly, it may be desirable to increase the yield of sorted sperm cells affording rare or valuable genetic material. Alternately, the number of sex selected sperm cells in a 2 million sex sorted sperm are included in each straw, then 1000 units of cryopreserved sexed semen can be produced as well.

With respect to yield in the context of flow cytometry, and specifically the analysis and separation of sperm cells conventional means to increase yield typically takes the form of increasing the rate at which sperm cells are entrained in the fluid stream without an increase in the rate of flow of the fluid stream which has a value fixed due to the limitation as to the amount of pressure which can be utilized without impairing the viability or fertility of the sperm cells or other particles analyzed. However, as above discussed, this conventional approach has the disadvantage of increased co-incidence (more than one sperm cell in one droplet formed such droplets either discarded or contributing to a decrease in purity of the collected sperm cell populations). Because the half-life of sperm cells collected is limited, and because there did not exist prior to the instant invention a method of increasing yield without with out increasing co-incidence, it has been conventional practice to increase the number of sperm cells per unit volume of the fluid stream and sacrifice a certain portion of the sperm cells to co-incidence events or reduce the purity of the collected populations of sperm cells, or both.

Speed in the context of flow cytometry can mean the rate at which a plurality of particles are analyzed or the rate at which particles are separated based upon a differentiable characteristic and collected such as the occurrence of an X-chromosome bearing sperm cell or a Y-chromosome bearing sperm cell.

In the non-limiting example of sperm sorting, where separation of sperm cells into X-chromosome bearing and Y-chromosome bearing populations occurs continuously without interruption, speed of sort translates into more saleable units of sorted sperm cells per unit time. Since many costs are fixed, higher sorting speeds contribute to significantly higher profits, or respectively, ability to serve lower priced markets with higher volumes of sorted sperm. Additionally, a higher sort rate of sperm cells can correspond to a lesser amount of time in which sperm cells are held prior to freezing and this can result in greater motility, viability or fertility of the sorted sperm cells.

One way to increase the overall speed of sorting is to increase the pressure on the sheath fluid source (also referred to as a fluid source in the context of the generic invention) and particle source to increase the corresponding flow rates of the sheath fluid and the sample fluid, while keeping their proportional flow rates similar, and while increasing the drop formation rate. For the specific case of sperm cells, this is not possible with conventional flow cytometer means, since the higher pressures (and sheer forces) have been shown to damage or impair sperm cells. In addition, sort rates can be limited by the amount of time necessary for signal transfer and signal analysis. Both limits as to flow rate and analysis time have created an upper limit as to sort rate. Flow rates can be increased by using sample fluid conduits (also referred to as a particle injector), sheath fluid conduits and nozzle tips having a greater internal diameter (ID). However, the greater the ID of either of these elements contributes to a greater variation of centricity and higher CV which results in lower purity and lower yield. Accordingly, increasing diameter of these conduits may not afford a solution to the problem. The instant invention affords a solution to these problems with conventional microfluidic devices in general and specifically in regard to the analysis of particles by flow cytometry by providing a lubricious flow path for sorting cells with increased viability of sorted cells.

### II. SUMMARY OF THE INVENTION

Accordingly, a broad object of the invention can be to provide a lubricious microfluidic flow path system having a lubricious layer coupled to all or a part of the microfluidic conduits. Certain embodiments of the lubricious microfluidic flow path system provide in whole or in part microfluidic conduits having a lubricious layer which as compared to substantially identical conventional conduits allow an increase in the flow rate of a fluid stream in a microfluidic conduit without a corresponding increase pressure of the fluid stream, a decrease in pressure of the fluid stream without a corresponding decrease in the flow rate of the fluid stream, a decrease in the internal diameter of a microfluidic conduit without reduction in the flow rate of the fluid stream at a constant pressure, or the like.

A second broad object of the invention can be to provide a lubricious microfluidic flow path for a flow cytometer which can allow a simultaneous increase in particle analysis speed, purity of separated populations of particles based on differentiable particle characteristic(s), and yield.

A third broad object of the invention can be to provide a flow cytometer nozzle assembly which provides a nozzle, a sheath fluid conduit, and a sample fluid conduit each in whole or in part or in various permutations and combinations having a lubricious layer coupled to the internal substrate surface.

A fourth broad object of the invention can be to provide a flow cytometer nozzle assembly which provides sample fluid conduit having a lubricious layer coupled to the internal substrate surface which allows utilization of a sample fluid stream of greater flow rate without substantial increase in sample fluid stream pressure or a providing a sample fluid conduit having a lubricious layer which provides reduced internal diameter which allow utilization of a sample fluid stream without substantial reduction in sample fluid flow rates and without substantial increase in pressure.

A fifth broad object of the invention can be to provide a flow cytometer nozzle assembly which provides a nozzle having a lubricious layer coupled to the internal substrate surface which allows utilization of a fluid stream of greater flow rate without substantial increase in fluid stream pressure.

Naturally, further objects of the invention are disclosed throughout other areas of the specification, drawings, photographs, and claims.

### III. A BRIEF DESCRIPTION OF THE DRAWINGS

FIG. 1 shows a particular embodiment of the lubricious microfluidic flow path system.

FIG. 2 shows a particular embodiment of a viewable data representation generated by a particular embodiment of the lubricous microfluidic flow path system shown in FIG. 1.

FIG. 3 shows a side view of a particular embodiment of a nozzle assembly for a particular embodiment of the lubricious microfluidic flow path system shown in FIG. 1.

FIG. 4 shows a top view of a particular embodiment of a nozzle assembly for a particular embodiment of the lubricious microfluidic flow path system shown in FIG. 1.

FIG. 5 shows an end view of a particular embodiment of a nozzle assembly for a particular embodiment of the lubricious microfluidic flow path system shown in FIG. 1.

FIG. 6 shows an opposite end view of a particular embodiment of a nozzle assembly for a particular embodiment of the lubricious microfluidic flow path system shown in FIG. 1.

FIG. 7 shows a cross section 7-7 of FIG. 5 of a particular embodiment of a nozzle assembly for a particular embodiment of the lubricious microfluidic flow path system shown in FIG. 1.

FIG. 8 is a cross section 8-8 of FIG. 7 of a particular embodiment of a nozzle assembly for a particular embodiment of the lubricious microfluidic flow path system shown in FIG. 1.

FIG. 9 is an enlargement of a part of FIG. 7 which shows a portion of a nozzle of a nozzle assembly for a particular embodiment of the lubricious microfluidic flow path system shown in FIG. 1.

FIG. 10 is an enlargement of a part of FIG. 9 which shows a portion of a sample fluid conduit of a nozzle assembly for a particular embodiment of the lubricious microfluidic flow path system shown in FIG. 1.

FIG. 11 is an enlargement of a part of FIG. 9 which shows a portion of a sample fluid conduit having a lubricious layer of a nozzle assembly for a particular embodiment of the lubrious microfluidic flow path system shown in FIG. 1.

FIG. 12 provides a cross section view of a lubricious microfluidic conduit included as part of particular embodiments of a lubricious microfluidic flow path system.

FIG. 13 provides a cross section view of a particular embodiment of a nozzle assembly of the lubricious microfluidic flow path system shown in FIG. 1.

FIG. 14 provides a cross section view of a particular embodiment of a nozzle assembly of the lubricious microfluidic flow path system shown in FIG. 1.

FIG. 15 provides a cross section view of a particular embodiment of a nozzle assembly of the lubricious microfluidic flow path system shown in FIG. 1.

FIG. 16 provides a cross section view of a particular embodiment of a nozzle assembly of the lubricious microfluidic flow path system shown in FIG. 1.

FIG. 17 provides a graph which plots internal diameter of conduit against flow rate at constant pressure to provide a comparison of performance of a conventional conduit with particular embodiments of the inventive conduit having a lubricious layer coupled to the internal substrate surface.

FIG. 18 provides a cross section of a part of a conventional nozzle assembly of a flow cytometer.

FIG. 19 provides a cross section of a part of a particular embodiment of an inventive nozzle assembly of the particular embodiment of a microfluidic flow path system shown in FIG. 1.

FIG. 20 provides a cross section of a part of a particular embodiment of an inventive nozzle assembly of the particular embodiment of a microfluidic flow path system shown in FIG. 1.

FIG. 21 provides a graph which plots flow rate of a fluid stream against pressure to provide a comparison of performance of particular embodiments of the inventive conduit having a lubricous layer against conventional performance of conventional conduits.

FIG. 22 provides a cross section view of a particular embodiment of an inventive conduit having a lubricious layer for use with embodiments of a microfluidic flow path system.

### IV. DETAILED DESCRIPTION OF THE PREFERRED EMBODIMENTS

Generally, a lubricious microfluidic flowpath for a particle analysis device. Specifically, a flow cytometer which provides a lubricious flow path for the analysis and separation of particles.

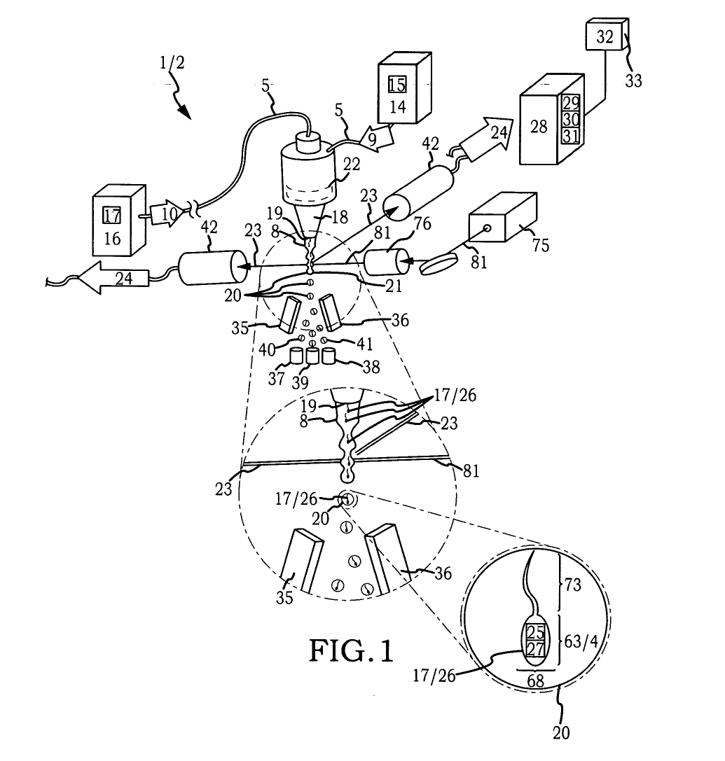

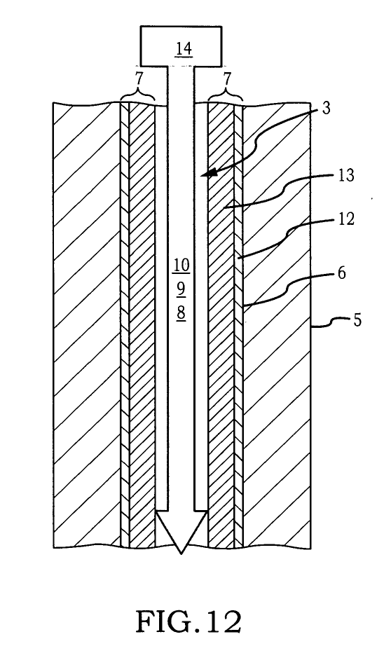

Now referring primarily to FIGS. 1, 7 and 12, which provides a non-limiting example of a lubricious microfluidic flow path system (1) in the form of flow cytometer (2) for the analysis of a plurality of particles (17). While the example provided by FIG. 1 is in the form of a flow cytometer (2), the term “lubricious microfluidic flow path system” for the purposes of this invention means any microfluidic device such as a chromatograph, flow cytometer, or the like, having a conduit (5) (see for example FIG. 12) at least a part of which provide(s) an internal substrate surface (6) configured to provide a flow path having a diameter of about one millimeter or less with a part or all of such internal substrate surface (6) of the conduit (5) coupled to a lubricious layer (7) sufficiently thick to reduce resistance to flow of a fluid stream (8) (which as to flow cytometer embodiments of the invention can include a sheath fluid stream (9) or a sample fluid stream (10)) within the conduit (5). The term “lubricious flow path” for the purposes of this invention means the flow path defined by the configuration of a lubricious layer (7) coupled directly or indirectly to the internal substrate surface (6) of a conduit (5). The term “lubricious layer” for the purposes of this invention means a layer, a film, a sheath or the like coupled directly or indirectly to the internal substrate surface (6) of a conduit (5) which increases lubricity of the internal substrate surface (6) to decrease resistance to flow of a fluid stream (8) within the conduit (5), or allows reduction of pressure of a fluid stream (8) within the conduit (5), or allows increased rate of flow of a fluid stream (8) within the conduit (5), or allows increased rate of flow of a fluid stream (8) which entrains a plurality of particles (17), reduces the turbulence of a fluid stream (8), minimizes alteration or impairment of a plurality of particles, or the like.

As one non-limiting example, a lubricious layer (7) can be achieved by utilizing a composition including a mixture of a high viscosity linear siloxane polymer with a reactive silicone polymer dissolved or diluted in a low viscosity siloxane polymer which acts as a carrier for the composition. A particular embodiment of the composition can provide a first low viscosity siloxane polymer having a viscosity of less than about 50 centistokes, a second high viscosity siloxane polymer having a viscosity of greater than about 1,000 centistokes and desirably greater than about 5,000 centistokes, and a reactive silicone polymer which is capable of crosslinking to form a crosslinked polymer network. The reactive silicone polymer can be cured by exposure to radiation such as UV light to form a crosslinked three-dimensional network which adheres to the substrate surface. At least a portion of the high viscosity linear siloxane polymer and the low viscosity siloxane polymer are contained within the crosslinked network structure as mobile liquid silicone oil which migrates to the surface of the layer to generate a continuously lubricious surface. A specific method of generating this composition is described by U.S. Pat. No. 7,332,227 to Hardman et al which is incorporated by reference herein.

As second non-limiting example, a lubricious layer (7) can be achieved by utilizing a composition which includes a silicone-epoxy copolymer mixed with a cationic photoinitiator that is dispersed with vinyl ether and a further secondary silicone component. The silicone-epoxy copolymers are organo-functional polydimethylsiloxane polymers where methyl groups are replaced with reactive organic moieties including acrylate, oxirane, or other readily polymerizable groups. A particular UV/EB curable silicone copolymer component of this type can be obtained from Rhodia Silicones, Rock Hill, S.C. under the trade name Silcolease. These radiation curable silicone copolymers available from Rhodia include, but are not limited to, Silcolease PC-675, Silcolease PC-670, Silcolease PC-600 and Silcolease PC-601. These radiation curable epoxy silicones are used with compatible iodonium-borate cationic photoinitiators such as onium type photocatalyst. It is contemplated within the scope of the invention that compatible onium salt photocatalyst utilized to catalyze the curing of the epoxy silicone in the process of the present invention may be any onium salt photocatalyst known within the art. These photocatalysts include but are not limited to the following bisaryliodonium salt catalysts: bis(dodecylphenyl) iodonium hexafluoroantimonate, bis(dodecylphenyl) iodonium hexafluoroarsenate and (4-octyloxyphenyl)(phenyl) iodinium hexafluoroantimonate. In one illustrative embodiment, the cationic photoinitiator materials are available from Rhodia Silicones, Rock Hill, S.C. These photoinitiators by Rhodia Silicones include but are not limited to Silcolease. PC-702, Silcolease PC-700 and Silcolease PC-702-30. In particular, Silcolease PC-702 is a 20 percent iodonium borate salt cationic photoinitiator in a diacetone alcohol carrier. A particular method of generating this composition is described in United States Patent Application No. 20050203201, hereby incorporated by reference herein.

As a third non-limiting example, a lubricious layer (7) can be achieved by utilizing a composition having a solids content of between about 15% and about 25% and which comprises a water-based urethane dispersion, a dimethyl siloxane emulsion and a polyfunctional aziridine. A urethane dispersion having a solids content of between about 30% and about 50% in a solution comprising a mixture of water, N-Methyl-2-pyrrolidone (CAS#872-50-4) and triethylamine (CAS#121-44-8). Such a dispersion can be obtained from Permuthane, Peabody, Mass. as UE41-222. A dimethyl siloxane can be obtained from Dow Corning Corporation, Midland, Mich. as Q2-3238. This is available neat and can be subsequently combined with water to form an emulsion having approximately 15% dimethyl siloxane. A polyfunctional aziridine is added to the solution (available from Permuthane, Peabody, Mass. as KM10-1703). A particular embodiment of the coating formulation has a solids content of approximately 17% upon application and comprises a mixture containing approximately 42.55% UE41-222 urethane dispersion, 12.77% Q2-3238 siloxane dispersion, 2.13% KM10-1703 polyfunctional aziridine and 42.55% distilled water. The formulation can be made by mixing the siloxane emulsion with the distilled water and subsequently adding the urethane dispersion. This is then mixed in a capped glass container with a magnetic stirrer until all parts are thoroughly mixed. The crosslinking agent is subsequently added to the solution just prior to application of the coating upon a surface. The addition of the crosslinking agent just prior to application of the coating prevents the urethane from crosslinking only with itself and thereby allows a sufficient carboxyl group density within the coating for crosslinking with the surface to be coated as described by U.S. Pat. No. 5,025,607, hereby incorporated by reference.

As a fourth non-limiting example, a lubricious layer (7) can be achieved by utilizing a composition comprising a multifunctional polymer or polymer combination, a hydrophilic polymer, colloidal metal oxide and a crosslinker as described by United States patent application 200330203991, hereby incorporated by reference. The multifunctional polymeric carrier can be a modified polymeric urethane, urea, ester, ether, carbonate, vinyl, acrylic, methacrylic, alkyd, acrylamide, maleic anhydride, an epoxy prepolymer and related polymers or a combination thereof. The hydrophilic organic monomer, oligomer, prepolymer or copolymer can be derived from vinyl alcohol, N-vinylpyrrolidone, N-vinyl lactam, acrylamide, amide, styrenesulfonic acid, combination of vinylbutyral and N-vinylpyrrolidone, hydroxyethyl methacrylate, acrylic acid, vinylmethyl ether, vinylpyridylium halide, melamine, maleic anhydride/methyl vinyl ether, vinylpyridine, ethyleneoxide, ethyleneoxide ethylene imine, glycol, vinyl acetate, vinyl acetate/crotonic acid, methyl cellulose, ethyl cellulose, carboxymethyl cellulose, hydroxyethyl cellulose, hydroxypropyl cellulose, hydroxymethyl ethyl cellulose, hydroxypropylmethyl cellulose, cellulose acetate, cellulose nitrate, starch, gelatin, albumin, casein, gum, alginate, hydroxyethyl (meth)acrylate, hydroxypropyl (meth)acrylate, ethylene glycol (meth)acrylates, N-alkyl (meth) acrylamides, N,N-dialkyl (meth)acrylamides, N-hydroxyalkyl (meth)acrylamide polymers, N,N-dihydroxyalkyl (meth)acrylamide polymers, ether polyols, polyethylene oxide, polypropylene oxide, poly(vinyl ether), alkylvinyl sulfones, alkylvinylsulfone-acrylates or a combination thereof. The multifunctional aqueous colloidal metal oxide can be derived from a metal selected from the group consisting of aluminum, silicon, titanium, zirconium, zinc, tin or silver, related colloidal metal oxides and a combination thereof. The multifunctional crosslinker can be a multifunctional aziridine, carbodiimide, oxirane, alcohol, glycydyl ether, glycidyl ester, carboxyl compound, amine, epoxide, vinyl sulfone, amide, allyl compound and related hardener, their related prepolymeric resins and combinations thereof.

As a fifth non-limiting example, a lubricious layer (7) can be achieved by utilizing a composition comprising comprise a mixture of about 0.32 g. of an aromatic polyisocyanate adduct based on toluene diisocyanate and dissolved in propylene glycol monomethyl acetate and xylene having an NCO content of about 10.5% and a molecular weight of about 400 available as DESMODUR DB 60 from Bayer Corporation; and about 0.67 g. of a solvent-free, saturated polyester resin (polyol) available as DESMOPHEN 1800 from Bayer Corporation; and about 0.91 g. of polyethylene oxide available as POLYOX having a molecular weight of about 300,000 from Union Carbide Corp; and about 76.97 g. acetonitrile; and about 21.82 g. THF; and about 2.02 g. 3-isocyanyopropyltriethoxysilane available as UCT 17840-KG from United Chemical Technologies, Bristol, Pa.

As sixth non-limiting example, a lubricious layer (7) can be achieved by utilizing a composition comprising a solvent mix of between about 15 g and about 40 g polyvinylpyrrolidone, between about 15 g and about 40 g of aqueous polyurethane dispersion, and between about 0.5 g and 2.0 g aziridine cross linker, the mixture diluted sufficiently with water to flow through the conduit (5) being treated.

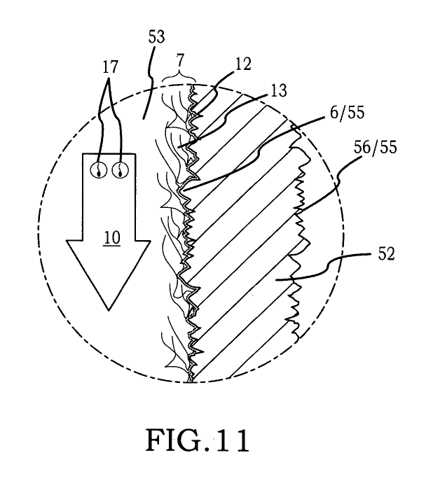

A seventh non-limiting example (refer also to FIGS. 11 and 12), a lubricious layer (7) can be achieved by utilizing a composition which comprises a first binding layer (12) which couples to the internal substrate surface (6) of the conduit (5) and a second bound layer (13). The first binding layer (12) can comprise but is not limited to a polymeric composition such as a diene compound, admixed with either magnesium oxide alone or magnesium oxide together with cobalt ion or manganese ion, or the like. The polymeric composition can be sufficiently diluted with solvent to flow through the conduit (5) and can be incubated for a period of time between about 5 seconds and about 60 seconds; although lesser or greater periods of time can be utilized to achieve a particular thickness of the lubricious layer (7). Excess polymeric composition can be expelled from the conduit (5) under pressure and further with vibration. An amount of solvent can be passed through the conduit (5) to relieve the conduit (5) of any remaining unbound polymeric composition. The excess solvent residual within the conduit (5) can be brought to dryness by flowing an amount of gas through the conduit with elevated temperature or without elevated temperature or with or without reduced pressure to provide the first binding layer (12). Where a diene compound is utilized, the first binding layer (12) can afford a plurality of primary hydroxyl groups to which can be reacted with a composition to produce the second bound layer (13). As an alternative embodiment, the first binding layer (12) can generated using an isocyanate or isocyanate/polymer blend and the second bound layer (13) can be generated using a hydrophilic polymer such as polyvinyl pyrrolidone or polyethylene oxide. The resulting first binding layer (12) and the second bound layer (13) can cured at a temperature of about 115° centigrade (“° C.”) to about 170° C.

It is not intended that these examples of achieving a lubricous layer (7) on an internal substrate surface (6) of a conduit (5) be limiting with respect the numerous and varied compositions which can be used to provide the lubricous layer (7) described herein and as further described for example by U.S. Pat. Nos. 4,192,827; 3,961,379; 4,229,551; 4,254,239; 4,350,791; 4,373,009; 4,408,026; 4,990,357; 5,026,607; 5,041,100; 5,061,424; 5,084,315; 5,091,205; 5,160,790; 5,250,620; 5,283,298; 5,29,505; 7,332,227; 7,220,491; 6,656,517; 6,509,098; 7,338,620; 7,321,012; United States Patent Application Nos. 20050203201 and 2003014227; and Patent Cooperation Treaty Publication Nos. WO/2006/032043 and WO/2005/025633, each hereby incorporated by reference.

The compositions which can be used to achieve the lubricious layer (7) can be applied to the internal substrate surfaces (6) of conduits (5) using any one or a combination of methods, such as: aerosol deposition, flowing, dipping, rolling, brushing, or the like. Understandably, application of the above-described composition(s) to the internal substrate surface (6) of a conduit (5) to produce a lubricious microfluidic flow path system (1) may preclude the use of certain application methods and may preclude the use of photocatalyzed compositions and photoinitiated methods to cure the composition.

Specifically, in regard to producing a lubricious microfluidic flow path system (1), certain compositions can provide flowable or can be made flowable through a conduit (5) having an internal diameter of less than one millimeter. Flowable compositions whether applied in the form of one composition to provide the lubricious layer (7) or in the form of a first binding layer (12) and a second bound layer (13) (or more layers) to provide the lubricious layer (7) are each typically flowed within the conduit (5) for a period of time between five seconds and 60 seconds; although greater or lesser periods of time may be utilized to achieve a desired thickness of the lubricious layer (7). A solvent can be flowed through the conduit (5) to relieve any unbound composition from within the conduit (5). The solvent can be dried by passing an amount of gas through the conduit (5). The lubricious layer (7) can be cured at ambient temperature or elevated temperature of about 115° centigrade (“° C.”) to about 170° C. at atmospheric or at reduced pressures or permutations and combinations thereof. The lubricious layer (7) achieved in this manner can have a thickness of between about 0.1 microns to about 100 microns or provide sufficient thickness to achieve a consistent measurable resistance to the flow of a fluid stream (8) between treated conduits (5) or consistent measurable pressure of the fluid stream (8) between treated conduits (5). In the context of a lubricious microfluidic flow path system (1) utilized for the analysis of particles, sufficient consistency with respect to achieving a particular resistance to flow of a fluid stream (8), pressure of a fluid stream (8) or the like can be achieved by steps which monitor increasing thickness of the lubricious layer (7) on the internal substrate surface (6). By measuring resistance or measuring pressure of the flowable composition during the period of application to the internal substrate surface (6) of the conduit (5) the flow characteristics of a plurality of conduits (5) can be standardized to a level suitable for use in the production of various embodiments of a lubricious microfluidic flow path system (1).

Understandably, one of the advantages of coupling a lubricious layer (7) to the internal substrate surface (6) of a conduit (5) which cannot be otherwise achieved can be to make less variable or make more consistent characteristics of the internal substrate surface (6) such as internal diameter, smoothness, lubricity, or the like) of the same or between different conduit(s) (5) which results in a greater consistency of the fluid stream (8) flow characteristics (such as laminar flow, pressure, resistance to flow, dispersion of a plurality of particles, or the like) in the same or between different conduit(s) (5) notwithstanding the additional advantages hereinafter described.

Accordingly, certain embodiments of invention can further include steps by which the viscosity of the composition can be fixed or substantially fixed and the flow rate of the composition within a particular conduit (5) can be fixed or substantially fixed at values which allow a selected pressure to be generated within the conduit (5) as the lubricious layer (7) achieves a desired thickness within the conduit (5). This approach to applying any one or more of the compositions to the internal substrate surface (6) of a conduit (5) can be useful because individual pieces of untreated conduit (5) can vary with respect to internal diameter and accordingly can generate different amounts of pressure with respect to the same fluid flowed at the same rate within different pieces of conduit (5). This difference in the amount pressure generated between individual pieces of conduit (5) can be reduced or the difference substantially eliminated by flowing the selected composition for the period of time necessary to achieve a lubricious layer (7) having a thickness which makes consistent the internal diameter of the lubricious flow path (3) or makes consistent the pressure generated within conduit (5) with respect to the same fluid flowed at substantially the same flow rates in a plurality of different conduits (5). Upon achieving a thickness in the lubricious layer (7) which makes consistent or reduces the variance in the amount of pressure between separate pieces of conduit (5), the flow of composition within the conduit (5) being treated can be interrupted and the remaining amount of composition within the conduit (5) ejected by flowing an amount of gas or an amount of solvent through the lubricious flow path (3) of the conduit (5). Any remaining solvent can be brought to dryness and the lubricious layer (7) cured. The lubricious layer (7) achieved can provide a lubricious flow path (3) which generates as between different pieces of treated conduit (5) similar pressure in a flow of fluid (8) of similar viscosity and similar flow rate.

Similarly, as to certain embodiments the invention can further include steps by which the viscosity of the composition can be fixed or substantially fixed and the fluid supply pressure of the composition within a particular conduit (5) can be fixed or substantially fixed at values which allow a selected flow rate to be generated within the conduit (5) as the lubricious layer (7) achieves a desired thickness within the conduit (5). This approach to applying any one or more of the compositions to the internal substrate surface (6) of a conduit (5) can be useful because individual pieces of untreated conduit (5) can vary with respect to internal diameter and accordingly can generate different flow rates with respect to the same fluid pressure at the same fluid pressure within different pieces of conduit (5). This difference in the flow rates generated between individual pieces of conduit (5) can be reduced or the difference substantially eliminated by flowing the selected composition for the period of time necessary to achieve a lubricious layer (7) having a thickness which makes consistent the internal diameter of the lubricious flow path (3) or makes consistent the flow rate generated within conduit (5) with respect to the same fluid flowed at substantially the same fluid pressure rates in a plurality of different conduits (5). Upon achieving a thickness in the lubricious layer (7) which makes consistent or reduces the variance in the flow rate between separate pieces of conduit (5), the flow of composition within the conduit (5) being treated can be interrupted and the remaining amount of composition within the conduit (5) ejected by flowing an amount of gas or an amount of solvent through the lubricious flow path (3) of the conduit (5). Any remaining solvent can be brought to dryness and the lubricious layer (7) cured. The lubricious layer (7) achieved can provide a lubricious flow path (3) which generates as between different pieces of treated conduit (5) similar pressure in a flow of fluid (8) of similar viscosity and similar flow rate.

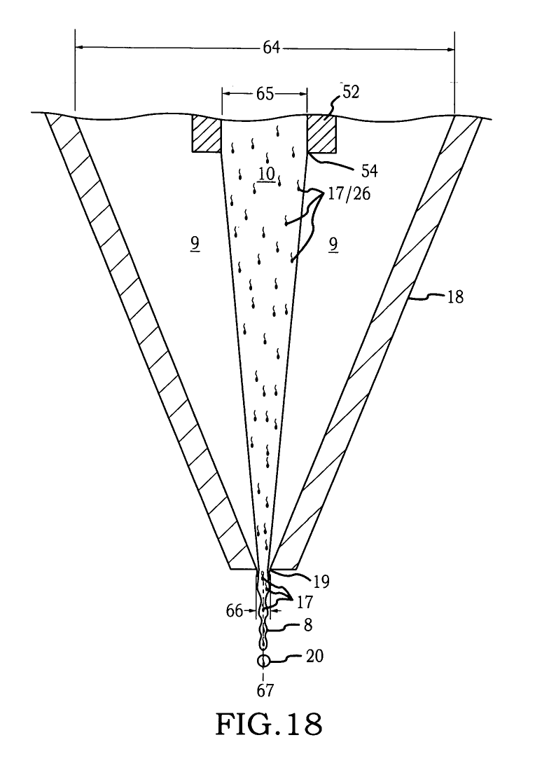

Again referring primarily to FIGS. 1, 7 and 12, a flow cytometer (2) having a lubricious flow path (3) can provide a fluid source (14) which supplies a sheath fluid (15) to establish a sheath fluid stream (9). A particle source (16) can entrain a plurality of particles (17) in a sample fluid stream (10). The sample fluid stream (10) entraining the plurality of particles (17) joins the sheath fluid stream (9) in the nozzle (18) of the flow cytometer (2) as coaxial laminar flow with the sample fluid stream (10) surrounded by the sheath fluid stream (9) (see for example FIGS. 18-20). The coaxial laminar flow exits the nozzle orifice (19) and can be established below the nozzle (18) as a fluid stream (8) entraining the plurality of particles (17).

The nozzle (18) can be made responsive to an oscillator (22) (see FIG. 1 broken lines). Oscillation of the nozzle (18) can perturb the fluid stream (8) to establish a steady state oscillation of the fluid stream (8). One non-limiting example of an oscillator (22) capable of perturbing the fluid stream (8) directly or indirectly by oscillation of the nozzle (18) is a piezoelectric crystal. The oscillator (22) may have an adjustable oscillation frequency that can be adjusted to perturb the stream at different frequencies (also referred to as the “drop drive frequency”). Steady state oscillation of the fluid stream (8) can be established in a condition such that droplets (20) are formed and break away from a contiguous part of the fluid stream (8). When the fluid stream (8) is established in this steady state fashion, a stable droplet break-off point (21) can be established. Increasing the drop drive frequency while fixing the fluid stream (8) velocity can result in a decrease in the drop radius. Fixing the drop drive frequency and increasing the fluid stream (8) velocity can result in an increase in drop radius. An increase in the drop drive frequency and an increase in the fluid stream (8) velocity can result in an increase in the number of droplets (20) per unit time with a fixed drop radius, as shown by the data set out in Table 1 below. By fixing the stream velocity and increasing the drop drive frequency, a reduction in drop radius can be achieved, as shown by the data set out in Table 2 below.

TABLE 1

| | | Drop Drive | | | |
|---|---|---|---|---|---|
| | Stream | Frequency | Drop | Drop | % |
| | Velocity | (DDF) | Volume | Radius | change |
| PSI | (meters/sec) | (KHz) | (nL) | (μM) | in DDF |
| 40 | 23.40 | 63.10 | 1.43 | 69.84 | 100.0% |
| 45 | 24.82 | 66.92 | 1.43 | 69.84 | 106.1% |
| 50 | 26.16 | 70.50 | 1.43 | 69.84 | 111.8% |
| 55 | 27.44 | 73.95 | 1.43 | 69.84 | 117.3% |
| 60 | 28.66 | 77.29 | 1.43 | 69.84 | 122.5% |
| 65 | 29.83 | 80.43 | 1.43 | 69.84 | 127.5% |
| 70 | 30.96 | 83.50 | 1.43 | 69.84 | 132.3% |
| 75 | 32.04 | 86.41 | 1.43 | 69.84 | 136.9% |
| 80 | 33.09 | 89.23 | 1.43 | 69.84 | 141.4% |
| 85 | 34.11 | 92.00 | 1.43 | 69.84 | 145.8% |
| 90 | 35.10 | 94.65 | 1.43 | 69.84 | 150.0% |

TABLE 2

| | | Drop Drive | | | |
|---|---|---|---|---|---|
| | Stream | Frequency | Drop | Drop | % |
| | Velocity | (DDF) | Volume | Radius | change |
| PSI | (meters/sec) | (KHz) | (nL) | (μM) | in DDF |
| 40 | 23.40 | 63.10 | 1.43 | 69.84 | 100.0% |
| 40 | 23.40 | 66.92 | 1.35 | 68.49 | 106.1% |
| 40 | 23.40 | 70.50 | 1.28 | 67.31 | 111.7% |
| 40 | 23.40 | 73.95 | 1.22 | 66.25 | 117.2% |
| 40 | 23.40 | 77.29 | 1.17 | 65.28 | 122.5% |
| 40 | 23.40 | 80.43 | 1.12 | 64.42 | 127.5% |
| 40 | 23.40 | 83.50 | 1.08 | 63.62 | 132.3% |
| 40 | 23.40 | 86.41 | 1.04 | 62.90 | 136.9% |
| 40 | 23.40 | 89.23 | 1.01 | 62.23 | 141.4% |
| 40 | 23.40 | 92.00 | 0.98 | 61.60 | 145.8% |
| 40 | 23.40 | 94.65 | 0.95 | 61.01 | 150.0% |

As evidenced by the data set out in Table 1 and Table 2, the drop radius and the number of droplets (20) formed per unit time can be finely adjusted by alteration of fluid stream (8) velocity and drop drive frequency.

However, with respect to certain particles, such as sperm cells (26), there may be disadvantages in decreasing drop radius below a certain radius. As a non-limiting example, intact bovine sperm cells (26) include a sperm head portion (4) having a sperm head length (63) of between about 5.0 μm and a sperm head cross sectional diameter (68) of about 4.0 μm and a tail portion (73) having a length of about 75 μm (total length about 80 μm). However, between species and breeds of bovine mammals and other species of mammals and other non-mammalian species having sperm cells which can be analyzed and sorted by the methods described herein, the dimensions of the head portion (63) and tail portion (73) can vary substantially (between about 60 μm and about 190 μm). With respect to sperm cells which can be analyzed or sex sorted by the methods described herein, a decrease in the drop radius which allows a portion of the sperm cell to extend outside of the boundary of one or more droplets (20) can impair analysis and sex sorting by FACS by reducing the consistency of orientation of the sperm cells (26) in the corresponding droplets (20), the interaction between the plurality of droplets (20) and the fluid stream (8), the location of the droplet break off point, and other factors as above described related to the consistency of analysis and sex sorting as above described. Additionally, a decrease in the drop radius below a certain dimension can impair viability or fertility of sex sorted sperm cells. Viability for the purposes of this invention can be assessed by percentage post thaw motility and percentage intact acrosome, as further described below. A reduction in sperm cell viability means a reduction in the percentage of motile sperm cells and a reduction in percentage intact acromsomes. An increase in sperm cell viability means an increase in the percentage of motile sperm cells or an increase in percentage intact acromsomes, or both.

Accordingly, once the smallest drop radius for a particular species, breed, or lot of sperm cells (26) (or other cells for which the drop radius effects analysis, sort or cell function), the drop radius can be fixed at or above that radius. Therefore, to obtain an increase in droplets (20) per unit time the stream velocity of the fluid stream (8) (sheath fluid stream (9) and sample fluid stream (10)) and the drop drive frequency must each be increased to provide a stable droplet break off point (21). The increase in the stream velocity of the fluid stream (8) results in an increase in pressure of the fluid stream (8) which can be achieved using particular embodiments of the invention without a reduction in viability or fertility of sex sorted sperm cells (26) and in certain applications the viability or fertility of sex sorted sperm cells (26) can actually increase, as shown by the examples set forth below.

The fluid stream (8) in steady state oscillation can be interrogated with one or more light beams (81) such as one or more a laser beams emitted from a light emission source (75). The one or more light beams (81) can pass through a beam shaping optics (76) to configure the shape of the one or more light beams (81) and focus the light beams on the fluid stream (8). An amount of light (23) emitted or reflected from the interrogated one of the plurality of particles (17) in the fluid stream (8) can be received by a photoreceiver (42). The photoreceiver (42) converts the received amount of light (23) into a signal (24) (whether analog, analog converted to digital, or digital) which varies whether in frequency, amplitude, or both frequency and amplitude) based upon differences in at least one particle characteristic (25) among the plurality of particles (17). The plurality of particles (17) can be biological particles such as cells, sperm cells, organelles, chromosomes, deoxyribonucleic acids (DNA), ribonucleic acids (RNA), DNA fragments, RNA fragments, proteins, protein fragments, peptides, oligonucleotides, or the like, but can also include non-biological particles such as beads, styrene beads, or the like, or as mixtures of biological particles, mixtures of non-biological particles, or mixtures of biological and non-biological particles. The term “at least one particle characteristic” for the purposes of this invention means at least one part, component, or differentially modified part or component common to at least a portion of the plurality of particles (17) entrained in the fluid stream (8) which varies in kind or amount between the plurality of particles (17).

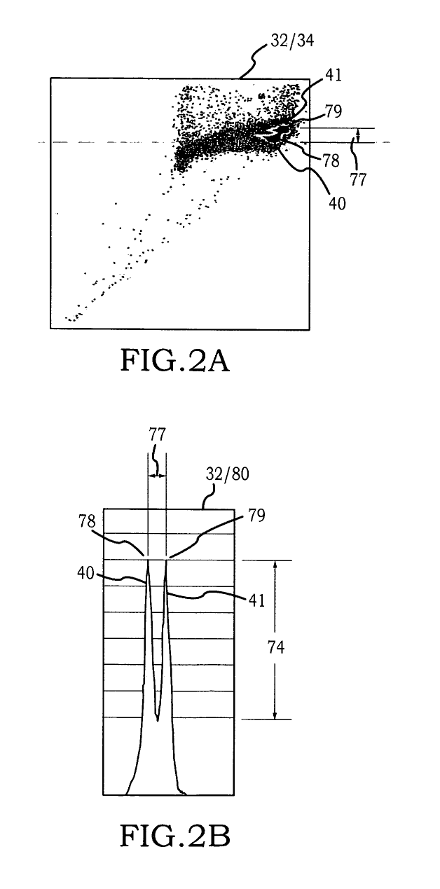

Now referring primarily to FIGS. 1 and 2, the flow cytometer (2) can further include a computer (28) which executes the functions of a particle analysis application (29) which in part provides a signal analyzer (30) which intermittently or continuously converts the signal (24) produced by interrogation of the fluid stream (8) into a data representation (31) of occurrence or detection of at least one particle characteristic (25) in the plurality of particles (17) interrogated. The data representation (31) can be continuously or intermittently displayed as a viewable data representation (32) (see for example FIGS. 2A and 2B) on a monitor (33) or updated upon elapse of a short interval of time such as 100 milliseconds.

Certain embodiments of the signal analyzer (30) can further function to establish parameters and timed events by which the plurality of particles (17) can be separated, parsed, divided, or sex sorted based upon the presence, absence, or amount of the at least one particle characteristic (25). Based on the above-described analysis of each of the plurality of particles (17) in the fluid stream (8), the droplets can be differentiated based on at least one particle characteristic (25) and separated by applying a charge (whether positive or negative) to each one of the droplets (20) analyzed and then deflecting the trajectory of each of the droplets (20) by passing the droplets (20) through a pair of charged plates (35)(36). The trajectory of the positively charged droplets can be altered for delivery to a first container (37) and the trajectory of the negatively charged droplets can be altered for delivery to a second container (38). Uncharged droplets are not deflected and can be delivered to a third container (39) or to a waste stream.

As one non-limiting example, the plurality of particles (17) can be a plurality of sperm cells (26) and the at least one particle characteristic (25) can be the amount of deoxyribonucleic acid (“DNA”) (27) contained in each of the plurality of sperm cells (26). The amount of DNA (27) can vary based upon whether the particular one of the plurality of sperm cells (26) contains an X chromosome or a Y chromosome. The X chromosome contains a greater amount of DNA (27) than the corresponding Y chromosome regardless of the male mammal or other animal from which the plurality of sperm cells (26) is obtained. The DNA (27) can be stained (with Hoescht dye or DNA minor groove binders such as bis-benzamides, oligocarboxamides, polyamides, peptide nucleic acids, locked nucleic acids, or the like) and in response to interrogation with a light beam (22) such as a laser beam can emit an amount of light (23). X chromosome bearing sperm cells (40) typically emit a greater amount of light than Y chromosome bearing sperm cells (41) because each X chromosome bearing sperm cell (40) contains a greater amount of stained DNA (27) than a Y chromosome bearing sperm cell (41). The photoreceiver (42) can convert the amount of emitted light (23) into a signal (24) which correspondingly varies based upon the difference in the amount of light (23) emitted by X chromosome bearing sperm cells (40) and Y chromosome bearing sperm cells (41) when passed through the light beam (22). With respect to the separation of a plurality of sperm cells (26), the separated sub-populations can include X chromosome bearing sperm cells (40) isolated in the first container (37) and Y chromosome bearing sperm cells (41) isolated in the second container (38). Sperm cells (26) can be obtained from any of a wide and numerous variety of male mammals including for example, a bovid, an ovis, an equid, a pig, a cervid, a canid, a felid, a rodent, a whale, a rabbit, an elephant, a rhinoceros, a primate, or the like, as well as from certain male non-mammal species such as a species of fish.

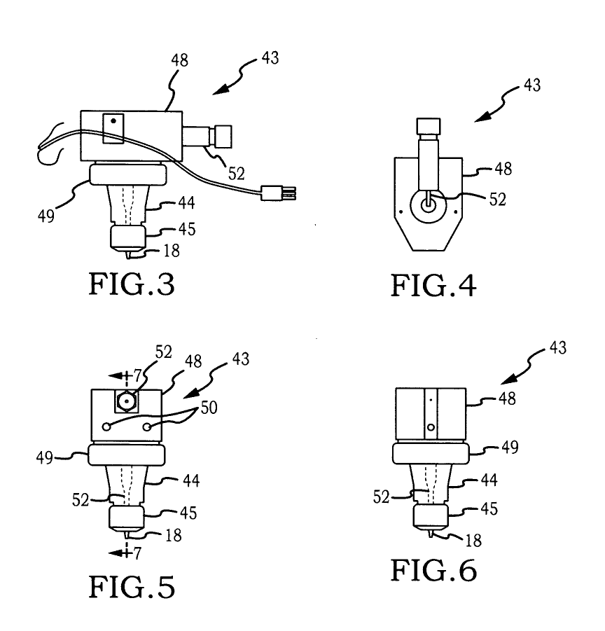

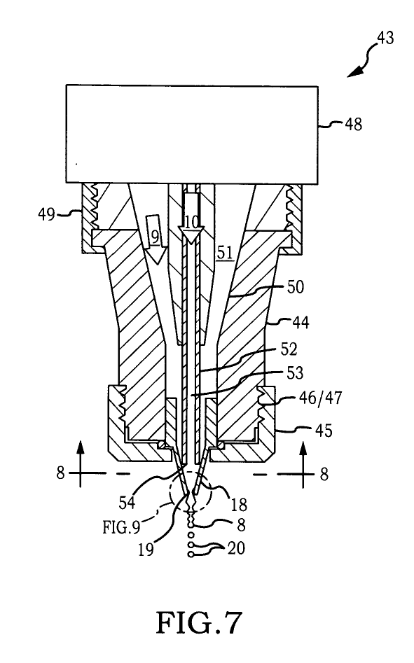

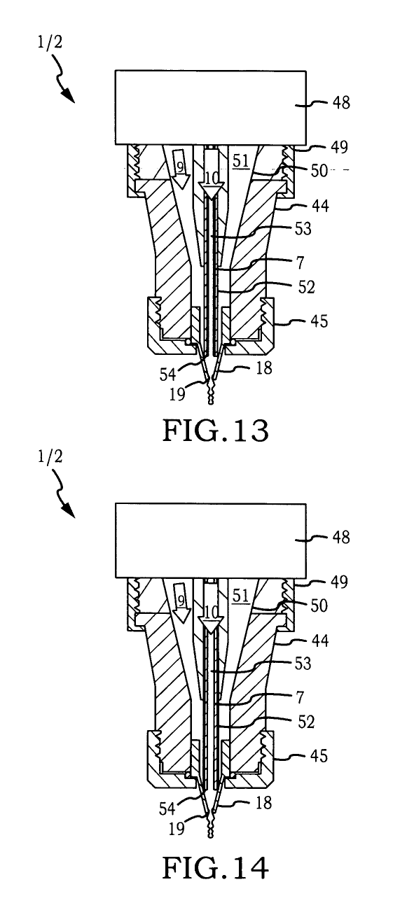

Now primarily referring to FIGS. 3-7, embodiments of a nozzle assembly (43) of a flow cytometer (2) can provide a lubricious flow path (3) of various configurations as further described below and as shown in FIGS. 13-16. The nozzle assembly (43) can include in part the nozzle (18) as above described which couples to a nozzle manifold (44). Securement of the nozzle (18) to the nozzle manifold (44) can be achieved by providing a nozzle retaining element (45) which engages the nozzle (18) and the nozzle manifold (44) to forcibly urge in fluidicly sealed engagement mateable surfaces of the nozzle (18) and the nozzle manifold (44). The particular embodiment of the nozzle retaining element (45) shown provides a nozzle retaining element spiral thread (46) which rotating engages a first nozzle manifold spiral thread (47) (see for example FIG. 7) to draw the nozzle retaining element (45) toward the nozzle manifold (44). Similarly, securement of the nozzle manifold (44) to a nozzle assembly housing (48) can be achieved by providing a nozzle manifold retaining element (49) which engages the nozzle assembly housing (48) to forcibly urge in fluidicly sealed engagement mateable surfaces of the nozzle manifold (44) and the nozzle manifold (44).

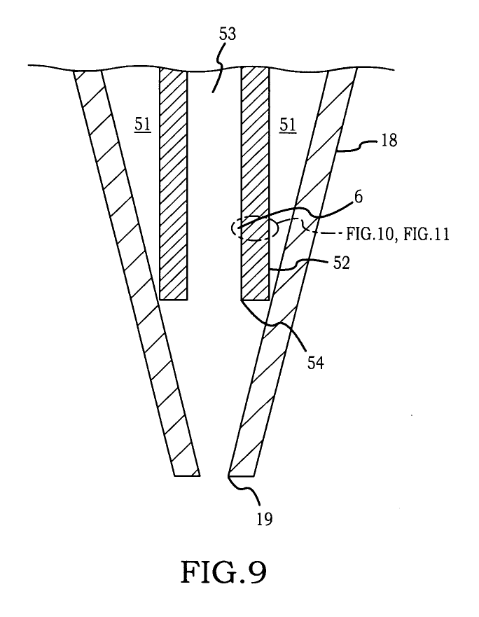

Now referring specifically to FIGS. 5, 7 and 9, a pair of sheath fluid conduits (50) provide a sheath fluid flow path (51) fluidicly coupled to the inside space defined by the nozzle manifold (44) sealably engaged to the nozzle (18). Similarly, a sample fluid conduit (52) provides a sample fluid flow path (53) fluidicly coupled to the inside space defined by the nozzle (18) sealably engaged to the nozzle manifold (44). The sample fluid conduit (52) extending downwardly inside the nozzle manifold (44) can have a configuration which provides a sample injection orifice (54) substantially coaxially aligned with the nozzle orifice (19) (see for example FIGS. 7 and 9).

Now specifically referring to FIG. 7, which provides cross section 7-7 of a nozzle assembly (43) shown in FIG. 5 of a flow cytometer (2) which shows sealed engagement of the nozzle manifold (44) and the nozzle assembly housing (48) by use of a nozzle manifold retaining element (49) and further shows the sealed engagement of the nozzle (18) with the nozzle manifold (44) with the nozzle retaining element (45). The inside space defined by sealed engagement of the nozzle (18) with the nozzle manifold (44) provides the sheath fluid path (51) above described which surrounds the sample fluid flow path (53) below the sample injection orifice (54). The resulting fluid stream (8) can exit the nozzle orifice (19) as a coaxial laminar flow with the sample fluid stream (10) at the center surrounded by the sheath fluid stream (9), as further described below.

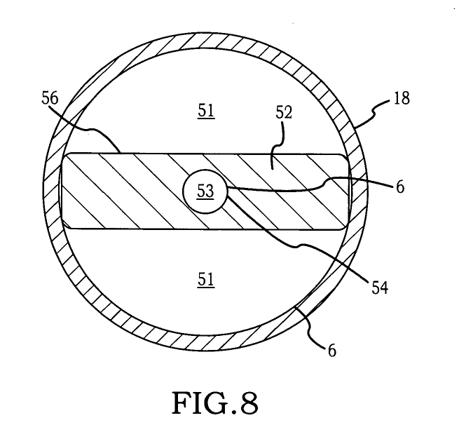

Now referring specifically to FIG. 8, which provides cross section 8-8 as shown in FIG. 7 which shows the relationship between the sample fluid conduit (52) and the nozzle (18) at the location of the sample injection orifice (54) of a particular embodiment of a nozzle assembly (18). As to certain embodiments of the sample fluid conduit (52) the internal substrate surface (6) can be substantially circular in cross section and the internal substrate surface of the nozzle (18) can also be substantially circular in cross section. The external surface ( ) of the sample fluid conduit (52) can configured to transition between a substantially circular cross section a distance above the sample injection orifice (54) to a substantially rectangular cross section at the sample injection orifice (54), as shown in the cross section view of FIG. 8.

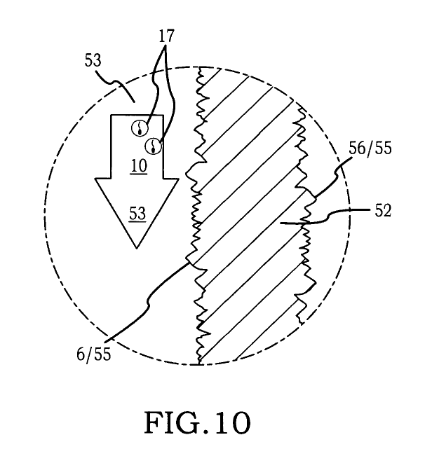

Now referring specifically to FIGS. 9 and 10, which provides an enlarged view of a part of FIG. 7 which shows the relationship of a part of the sample fluid conduit (52) to a part of the nozzle (18). A portion of the internal substrate surface (6) of the sample fluid conduit (52) is further enlarged in FIG. 10. As shown by FIG. 10, the internal substrate surface (6) of the sample fluid conduit (52) which defines the configuration of the sample fluid flow path (53) can be have surface irregularities (55). Similarly, the internal substrate surface (6) of the nozzle manifold (44) and the external substrate surface (56) of the sample fluid conduit (52) and the internal surface (6) of the nozzle (18) can also have surface irregularities (55). The surface irregularities (55) within the sample fluid conduit (52) can contribute to increased resistance to the flow of the sample fluid stream (10) and the plurality of particles (17) within the sample fluid stream (10). Additionally, the surface irregularities (55) can make adherence of some of the plurality of particles (17) or parts of the plurality of particles (17) or other materials more likely and can reduce random distribution of the plurality of particles within the sample fluid stream (10), or can increase turbulence in the sample fluid stream (10), or both. Similarly, surface irregularities (55) on the internal substrate surface (6) of the pair of sheath fluid conduits (50), the nozzle manifold (44), the nozzle (18) and at the nozzle orifice (19) can increase resistance to flow of the sheath fluid stream (9), increase turbulence, and reduce the number of droplets (20) which can be established and break off from the fluid stream (8) and reduce the incidence of entraining one of the plurality of particles (17) in each of the droplets (20), or increase co-incidence, as above-discussed. Surface irregularities (55) can also include surface irregularities (55) generated by polishing or electroplating the internal substrate surface (6) of the sample conduit (52). One non-limiting example of a surface irregularity (55) which can occur as result of electroplating or polishing a sample fluid conduit (52) is generation of a sample fluid injection orifice (54) have sufficiently lesser internal diameter than the internal diameter of the adjacent portion of the sample fluid conduit (52). This difference can dramatically reduce the performance of a flow cytometer (2)

Now referring primarily to FIG. 11, which shows a portion of the internal substrate surface (6) of the sample fluid conduit (52) further enlarged to show a lubricious layer (7) coupled to the internal substrate surface (6) of the sample fluid conduit (52). The particular embodiment of the lubricious layer (7) shown provides a first binding layer (12) and a second bound layer (13) as above described (although embodiments may provide the lubricious layer (7) as a single film or coat). The lubricious layer (7) functions to separate the sample fluid stream (10) and any of the plurality of particles (17) entrained in the sample fluid stream (10) from the surface irregularities (55). The sample fluid stream (10) and any of the plurality of particles (17) entrained can be engaged with the lubricious layer (7) which functions to reduce resistance to flow of the sample fluid stream (10), increase the likelihood of a random distribution of the plurality of particles (17) within the sample fluid stream (10), reduce the likelihood of adherence of the plurality of particles (17) with internal substrate surface (6) of the sample fluid conduit (52), and reduce turbulence within the sample fluid stream (10).

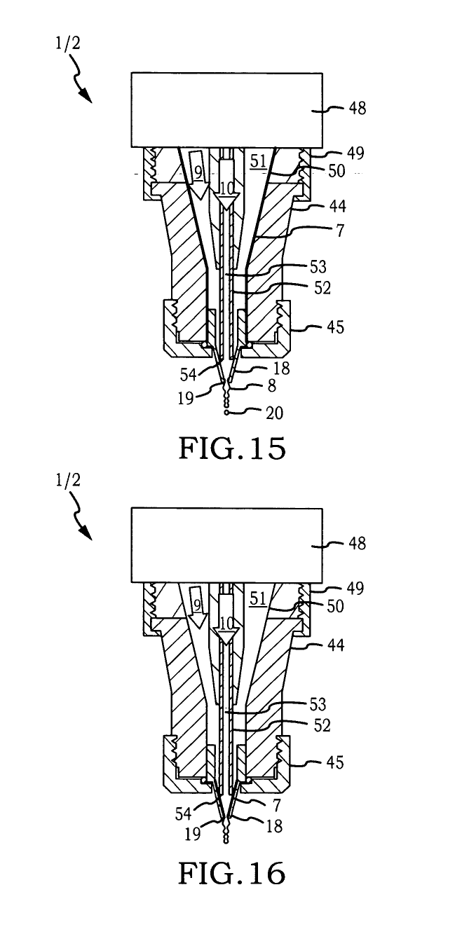

Now referring primarily to FIGS. 13-16, which show the relevant parts of certain embodiments of a lubricious microfluidic flow path system (1) for a flow cytometer (2) each embodiment providing a lubricious layer (7) coupled to certain surface(s) of the nozzle manifold (44) (FIG. 15), the sample fluid conduit (52) (FIGS. 13 and 14), or the nozzle (FIG. 16) of the nozzle assembly (43) of the flow cytometer (2). Now referring primarily to FIG. 13, the lubricious layer (7) can be coupled to a part or all of the internal substrate surface (6) of the sample fluid conduit (52). With respect to certain embodiments of invention, the lubricious layer (7) can be coupled to the portion of the internal substrate surface (6) of the sample fluid conduit (52) as shown in FIG. 13 or can be coupled to the entire length of the sample fluid conduit (52) from the sample injection orifice (54) to the particle source (16). Now referring primarily to FIG. 14, the lubricious layer (7) can be coupled to the external substrate surface (56) of the sample fluid conduit (52). The embodiment of the invention shown by FIG. 14, shows only a part of the external substrate surface of the sample fluid conduit (52) proximate to the sample injection orifice (54) coupled with the lubricious layer (7); although this does not preclude embodiments of the invention which couple the lubricious layer (7) to all external substrate surfaces of the sample fluid conduit (52) which fluidly engage the sheath fluid stream (9) within the nozzle manifold (44). Now referring to FIG. 15, the lubricous layer (7) can be coupled to the internal substrate surface (6) of the nozzle manifold (44). While the example shown couples the lubricious layer (7) to the entire internal substrate surface (6) of the nozzle manifold (44), this does not preclude coupling the lubricious layer (7) to only a portion of the nozzle manifold (44). Now referring to FIG. 16, an embodiment of the invention is shown which couples the lubricious layer to the internal substrate surface (6) of the nozzle (18). While these particular embodiments of the invention are shown as examples, this is not meant to limit the invention to these specific examples. Rather, a sufficient number of examples of the invention are provided to allow a person of ordinary skill to make and the use the numerous and varied embodiments of the invention whether or not specifically shown. Other embodiments of the invention can provide a lubricious layer (7) coupled to at least a portion of the substrate surface of the nozzle assembly (43) which define the sheath fluid flow path (51) or the sample fluid flow path (53), or both, whether the internal substrate surface or the external substrate surface of the sample fluid conduit (52), internal substrate surface of the nozzle (18), or the internal substrate surface of the nozzle manifold (44), or a combination of two or more or all of the embodiments shown in FIGS. 13-16 which can provide a lubricious layer (7) coupled to a part of one, to one, or all of the internal and external substrate surfaces of the nozzle manifold (44), nozzle (18), or sample fluid conduit (52) of a flow cytometer (2). Understandably, because a variety of flow cytometers, flow sort devices, and flow analysis devices can be modified in accordance with the invention to provide a lubricious microfluidic flow path system (1), embodiments of the invention can couple the lubricious layer (7) to a sufficient amount of the fluidicly engaged internal or external substrate surfaces to enhance a particular function, to increase overall performance, or yield a purified plurality of particles (17) which retain an increased level of a particular function or have overall increased viability.

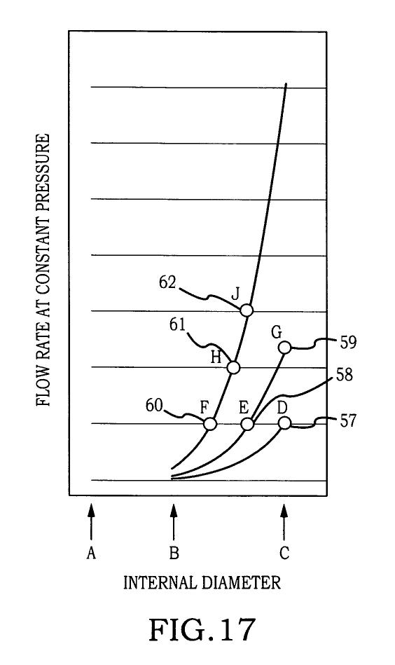

Now referring primarily to FIG. 17, which provides a graph which plots flow rate of at constant pressure of sample fluid stream (10) against increasing internal diameter of a sample fluid conduit (52) of a flow cytometer (2). Although not limiting, a conventional sample fluid conduit (42) for a flow cytometer (2) and other microfluidic devices such as chromatographs can be produced by the method of extrusion. Extrusion methods for producing a sample fluid conduit (52) for flow cytometers (2) and other conduits for microfluidic devices are typically performed using a molten material of high temperature polymer, such as PEEK (polyethyl ether ketone) or malleable metal such as stainless steel. Fluctuations in extrusion temperature, extrusion rate, or the like results in an internal substrate surface (6) which exhibits surface irregularities (55) as above described to a greater or lesser degree depending upon the extrusion conditions. The conventional method of extrusion leaves the internal substrate surface (6) of sample fluid conduit(s) (52) provided with conventional flow cytometers (2) with substantial surface irregularities (55) which can vary from extrusion to extrusion.

The internal substrate surfaces (6) of conventional extruded sample fluid conduit(s) (52) or other conduits can be “polished” to alter the configuration of the surface irregularities (55). One non-limiting method for polishing an untreated internal substrate surface (6) of a conduit is to pass a slurry containing fine hard particles such as synthetic diamond, through the flow path of the conduit resulting in a polished internal substrate surface. Another non-limiting method of “polishing” a internal substrate surface of a conduit can be to “electroplate” the untreated internal substrate surface (6) of the conduit. For example, by passing a solution of metal ions through a sample fluid conduit (52) and applying a voltage to the sample fluid conduit (52) ions can be reduced into non-ionic metal deposits on the internal substrate surface (6) of the sample fluid conduit (52). The disadvantages of polishing and electroplating can be that the treated surfaces of conduits can have a slightly larger internal diameter and have the internal substrate surface (6) impregnated with an amount of the polishing material as to the former method and requires an electrically conductive material in regard to the later. Finally, the internal substrate surfaces of a conduit can be treated in accordance with the invention to provide a lubricious layer (7).

As shown in FIG. 17, the horizontal axis of the graph provides different internal diameters (ID) of a sample fluid conduit (52) of a flow cytometer (2) of between about zero microns (a micron equals 10⁻⁶ meters) (point A) and about 250 microns (point C). Point B corresponding to an ID of about 100×10⁻⁶ meters (100 microns). The vertical axis shows the flow rate by volume of the sample fluid stream (1) at constant pressure passing through the sample fluid conduit (52) per unit time. This measure also allows for calculation of the plurality of particles (17) in number passing through the sample fluid conduit (52), as determined using the particle concentration in the particle source (16) of the flow cytometer. The actual units and dimensions are omitted, since the flow rate is determined by ID and the length of the particle injector tube and the operating pressures used. Accordingly, although embodiments of the invention can utilize sample fluid conduit (52) having an ID between about 50 microns and about 250 microns after application of the lubricious layer (7) through which a sample fluid stream (10) can be passed at between about 5 pounds of pressure per square inch (“psi”) and about 90 psi, the principles shown by the plots and by the description herein may apply to conduits having other IDs utilized for the analysis and separation of particles by a variety of microfluidic devices other than flow cytometers (2).

The plot line which contains Point D (57) represents the flow rates of the sample fluid stream (10) which can be achieved utilizing a conventional untreated sample fluid conduit (52). The plot line which contains Point E (58) and Point G (59) represents the flow rates of the sample fluid stream (10) which can be achieved utilizing a polished sample fluid conduit (52) as above-described. The plot line which contains Points F (60), H (61) and J (62) represents the fluid flow rates of the sample fluid stream (10) which can be achieved utilizing a sample fluid conduit (52) having a lubricious layer (7) coupled to the entirety of the internal substrate surface (6).

As depicted in FIG. 17, the sample fluid conduit (52) having a polished internal substrate surface (6) may deliver the sample fluid stream (10) at a flow rate of about 2.5 fold (Point G) (59) for particular specified operating pressure than for an untreated sample fluid conduit (52) (Point D (57)). If a higher flow rate of the sample fluid stream (10) is not desired, then an identical flow rate of the sample fluid stream (10) can be achieved by use of a sample fluid conduit (52) having a polished internal substrate surface (6) with a smaller ID (Point E) (58)). Alternately, without altering the ID of the polished internal substrate surface (6), the flow rate of the sample fluid stream (10) represented by Point G (59) could be adjusted downward to a flow rate represented by Points D (57) and Point E (58), by lowering pressure of the sample fluid stream (10).

Again referring to FIG. 17, the sample fluid conduit (52) treated to provide a lubricious layer (7) as above described or as shown for example in FIG. 13 can provide a greater flow rate of the sample fluid stream (10) at certain pressures as compared to a sample fluid conduit (52) which provides a polished internal substrate surface (6) or a conventional untreated internal substrate surface (6). For example, even though a polished sample fluid conduit (52) can allow about 2.5 fold greater flow rate of the sample fluid stream (10) as compared to a conventional untreated sample fluid conduit (52), a sample fluid conduit (52) which provides a lubricious layer (7) as shown for example by FIG. 13 can allow about a three-fold greater flow rate as compared to the sample fluid conduit (52) which provides a polished internal substrate surface (6) (for example compare Point J (62) to Point E (58)). Accordingly, a sample fluid conduit (52) which provides a lubricious layer (7) coupled to the internal substrate surface (6) as shown for example in FIG. 13 can provide about a fifteen fold greater flow rate of the sample fluid stream (10) as compared to an internal substrate surface (6) of a conventional sample fluid conduit (52) which does not provide a lubricous layer (7).

Now referring primarily to Figures to FIGS. 17, 18, 19 and 20, the greater flow rates provided by the inventive sample fluid conduit (52) which provides a lubricious layer (7) can afford the advantage of providing a flow cytometer (2) having a sample fluid conduit (52) of lesser internal diameter than a conventional flow cytometer (2). As shown for example by FIG. 17, each of Points J (62), H (61) and F (60) have substantially lesser diameter than the sample fluid conduit (52) of a conventional flow cytometer (2) represented by Point D (57) but each can provide a greater flow rate of the sample fluid stream (10) at normal flow cytometer (2) operating pressures which are typically in the range of about 5 psi to about 60 psi and with regard to analysis of sperm cells (26) between about 20 psi and about 45 psi.

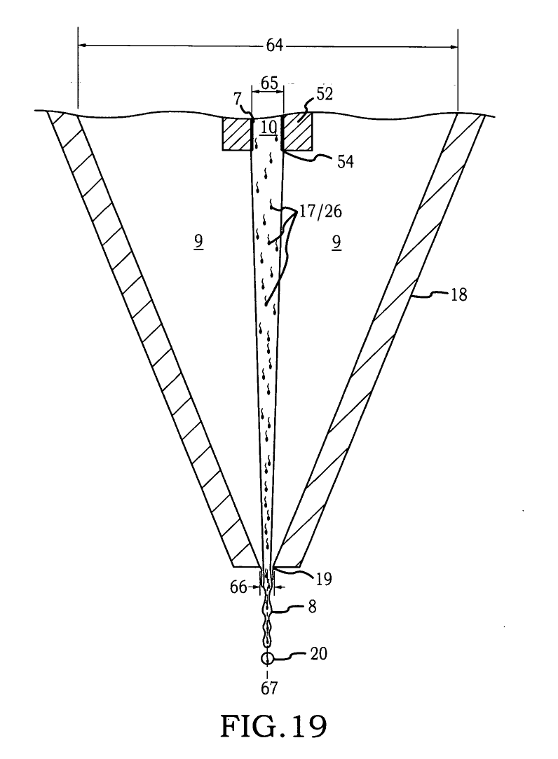

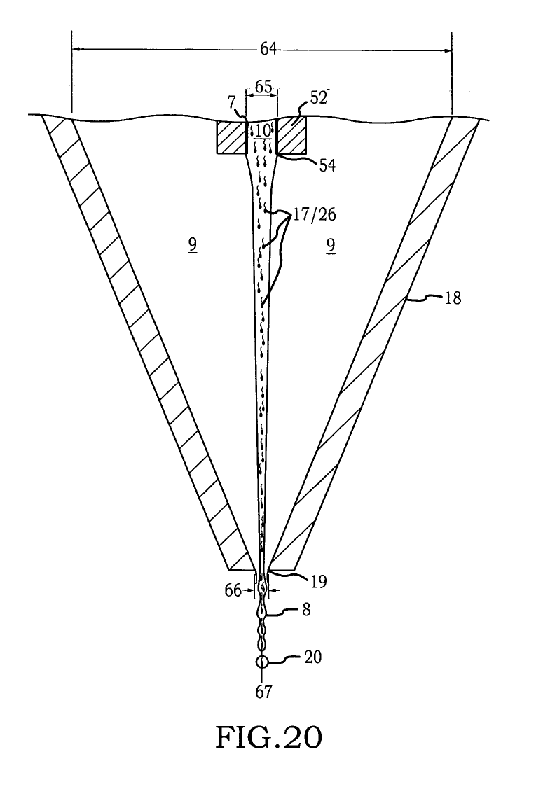

Now referring primarily to FIGS. 1, 18, 19, and 20, the figures provide an example of the advantages of utilizing the inventive sample fluid conduit (52) having a lubricious layer (7) to substantially increase centricity of the plurality of particles (17) relative to the longitudinal axis (67) of the fluid stream (8) exiting the nozzle (18). For the purposes of this invention the term “centricity” means centered or focused upon the longitudinal axis of the fluid stream (8) exiting from the nozzle orifice (18). Specifically, a greater level of centricity occurs as the center of volume of one of the plurality of particles (17) within the fluid stream (8) approaches coincidence with the longitudinal axis of the fluid stream (8) (or the average variance of the centers of volume of a plurality of particles (17) with the longitudinal axis of the fluid stream (8) decreases). With respect to a sperm cell (26) a greater level of centricity occurs as the center of volume of the sperm head (63) of the sperm cell (26) approaches coincidence with the longitudinal axis of the fluid stream (8). FIG. 18 provides an example of a plurality of particles (17) (such as sperm cells (26)) which have lesser centricity relative to the longitudinal axis (67) of the fluid stream (8) than the examples of the plurality of particles (17) (such as sperm cells (26)) provided by FIGS. 19 and 20.

Each of FIGS. 18, 19, and 20, describe a type of flow cytometer (2) in which the sample fluid conduit (52) has a configuration which locates the sample fluid conduit orifice (54) a distance from the nozzle orifice (19) in substantially coaxial relation inside the nozzle (18). This particular configuration of flow cytometer (2) allows the sample fluid stream (10) to engage the sheath fluid stream (9) in coaxial laminar flow. Each of the Figures describe the cross section of a cone or an epiliptical cone (see for example U.S. Pat. Nos. 6,263,745; 6,604,435; 6,782,768 as examples, each hereby incorporated by reference herein) corresponding to the internal fluid path of a downward pointing nozzle (18) of a flow cytometer (2) such as a MOFLO flow cytometer available from Beckman-Coulter; although the invention is not so limited. The nozzle internal diameter (64) at the location at which sample fluid stream (10) joins the surrounding sheath fluid stream (9) can be substantially identical for each nozzle (18) shown in each of FIGS. 18, 19, and 20. The nozzle orifice internal diameter (66) of the nozzle (18) can be identical for each nozzle (18) shown in each of FIGS. 18, 19, and 20. The distance between the sample fluid conduit orifice (54) and the nozzle orifice (19) can be substantially identical for each nozzle (18) shown in each of FIGS. 18, 19, 20.

Now referring primarily to FIG. 18, the sample fluid stream (10) exits the sample fluid conduit (52) to engage the sheath fluid stream (9) in substantially coaxial laminar flow. The sample fluid stream (10) can have a cross sectional area reduced by displacement of the surrounding sheath fluid stream (9) by the internal conical surface of the nozzle (18). The plurality of particles (17) (such as sperm cells (26)) remain entrained in the sample fluid stream (10) so long as there remains coaxial laminar flow of the sample fluid stream (10) surrounded by the sheath fluid stream (9). However, because each of the plurality of particles (17) can have a particle diameter substantially less than the sample fluid conduit internal diameter (65), a great variation in centricity of each of the plurality of particles (17) relative to the longitudinal axis (67) of the sample fluid stream (10) can occur. The variation in centricity for the plurality of particles (17) (such as sperm cells (26)) can have a measurable co-efficient of variation. As a non-limiting example, referring specifically to FIG. 18, if the nozzle internal diameter (64) is about 5,000 microns, and the sample fluid conduit orifice internal diameter (65) is about 250 microns, then the variation in centricity will be about 5% (250/5000). If the nozzle orifice internal diameter (66) is about 70 microns, then about 5% centricity variation corresponds to about 3.5 microns. Each of the plurality of particles (17) analyzed in the fluid stream (8) exiting a nozzle orifice internal diameter (66) of about 70 microns, will be within about 3.5 microns of the longitudinal axis (67) of the fluid stream (8). In the case of sperm cells (26), which can have a latitudinal cross-sectional diameter (68) of about 4 microns, this corresponds to a positional variation within the fluid stream of nearly 100% of the size of the sperm cell.

Now referring primarily to FIG. 19 which shows an inventive sample fluid conduit (52) having a lubrious layer (7) which allows use of a sample fluid conduit orifice internal diameter (65) reduced to about 40 microns; however the invention is not so limited. By use of a configuration of an inventive sample fluid conduit (52) having a lubricious layer (7), the variation in centricity as above determined can be reduced to about 0.8%. If the nozzle orifice internal diameter (65) is about 70 microns, then about 0.8% centricity variation corresponds to about 0.56 microns. Each of the plurality of particles (17) analyzed in the fluid stream (8) exiting a nozzle orifice internal diameter of about 70 microns, will be within about 0.56 microns of the longitudinal axis (67) of the fluid stream (8). In the case of sperm cells (26), which have a cross-sectional diameter of about 4 microns, this corresponds to a positional variation within the fluid stream (8) of about 0.14 of the size of each of the sperm cells (26) in latitudinal cross section (68) (see FIG. 1).

Use of a sample fluid conduit orifice (54) of reduced internal diameter (65) as shown in the example of FIG. 19 cannot be practically achieved without use of the inventive sample fluid conduit (52) having a lubricious layer (7) because a reduction in the sample fluid conduit internal diameter (65) as above-described at the same operating pressure using a conventional sample fluid conduit with an untreated internal substrate surface necessitates a substantial reduction in flow rate of the sample fluid stream (8) in some instance to as little as 3% of the flow rate achievable with the configuration of the sample fluid conduit (52) shown in FIG. 18. This substantial reduction in the flow rate of the sample fluid stream (8) correspondingly reduces the rate at which a plurality of particles (17) can be analyzed unless a sample fluid stream (8) at substantially greater pressure is utilized. However, increasing pressure of the sample fluid stream (8) may not be practical for applications in which the plurality of particles (17) comprise a plurality of biological particles such as cells, sperm cells, cell organelles, or macromolecular structures which must remain viable or fertile or both. Increased pressure of the sample fluid stream (10) and the sheath fluid stream (9) can reduce the viability, fertility or function of biological particles. As shown in FIG. 17, the flow rate of an inventive sample fluid conduit (52) having a lubricious layer (7) with a substantially reduced sample fluid conduit orifice internal diameter (65) (Point F (60)) can have flow rate a substantially similar to a conventional sample fluid conduit (52) having a sample fluid conduit orifice diameter (65) (Point D (57)) which is substantially greater. By providing a reduced diameter sample fluid conduit (52) having a lubricious layer (7), the sample fluid stream (10) entraining a plurality of particles (17) can be established at flow rate of between about 40% and about 100% of the flow rate of the conventional sample fluid conduit (52) having an untreated internal substrate surface which makes practical achievement of a substantially increased centricity of the plurality of particles (17) during operation of the flow cytometer (2) without substantial reduction in the analysis rate of the plurality of particles (17). Embodiments of the invention having a sample fluid conduit (52) with a lubricious layer (7), provide a reduced sample fluid conduit internal diameter which corresponds a gauge of between about 27 gauge and about 28 gauge, between about 28 gauge and about 29 gauge, between about 29 gauge and about 30 gauge, or between about 30 gauge and about 31 gauge. Reduction in the gauge of the sample fluid conduit (52) can result in a corresponding reduction in the cross section dimension of the sample fluid stream (10) surrounded by the sheath fluid stream (9). Accordingly, embodiments of the invention can provide a sample fluid stream (10) having a cross section dimension of between about 0.0080 inches and about 0.0075 inches, between about 0.0075 inches and about 0.0070 inches, between about 0.0070 inches and about 0.0060 inches, between about 0.0060 inches or about 0.0050 inches. Reduction of the sample fluid stream (10) can also allow use of a nozzle having a nozzle orifice (19) of reduced dimension of between about 70 μM and about 65 between about 65 μM inches and about 60 μM, between about 60 μM and about 55 μM, between about 55 μM and about 50 μM.

Now referring primarily to FIGS. 19 and 20 which afford substantially identical nozzle assembly (43) configurations, except that the flow rate of the sample fluid stream (10) is less than the flow rate of the sheath fluid stream (9). Conventionally, the analysis of sperm cells (26) results in a ratio of about one volume of the sample fluid stream (10) to about one hundred volumes of the sheath fluid stream (9) (about 1/100). Accordingly, the sample fluid stream (10) joins the sheath fluid stream (9) to generate a coaxial laminar flow at a flow rate of about one volume per unit time, while the sheath fluid stream flows into coaxial laminar flow surrounding the sample fluid stream (10) at a rate of about 100 volume units per unit time. When the flow rate of the sheath fluid stream (9) increases, while the flow rate of the sample fluid stream (10) remains the same or substantially the same, then an increase in centricity of each of the plurality of particles (17) can be achieved as the increase in flow rate of the sheath fluid stream (9) acts to further reduce the cross sectional area of the sample fluid stream (10) of the coaxial laminar flow. Each increase in the flow of the sheath fluid stream (9) relative to the sample fluid stream (10) decreases the cross sectional area of the sample fluid stream (10) within the fluid stream (8) which exits the nozzle orifice (19) and correspondingly increases the centricity of each of the entrained plurality of particles (17). The conventional untreated internal surface of the nozzle (18) can limit the flow rate at which the sheath fluid stream (9) can exit the nozzle orifice (19) because pressure within the nozzle (18) exceeds the practical limit at which viability of the plurality of particles can be maintained or because the laminar flow between the sample fluid stream (10) and the sheath fluid stream (9) cannot be maintained.

This problem can be addressed by utilizing the configuration of the inventive nozzle assembly (43) shown in FIG. 16 in which the internal substrate surface (6) of the nozzle (18) in whole or in part provides a lubricious layer (7) which terminates at the nozzle orifice (19). The lubricious layer (7) coupled to the internal substrate surface (6) of the nozzle (18) can act to allow a substantial increase in the flow rate of the sheath fluid stream (9) achievable as compared to a conventional nozzle (18) and a corresponding increase in centricity of each of the plurality of particles (17) relative to the longitudinal axis (67) of the fluid stream (8) not otherwise achievable for the reasons above described.

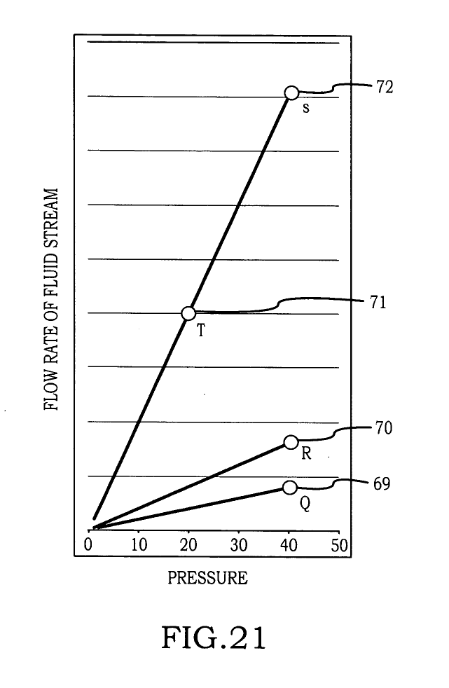

Now referring primarily to FIG. 21 which provides a graph which plots pressure against flow rate of the fluid stream (8) exiting the nozzle orifice (19) for certain configurations of the internal surface of the nozzle (18). The plot line which contains Point Q (69) represents flow rates achievable with an untreated internal substrate surface (6) of a conventional nozzle (18). The plot line which contains Point R (70) represents flow rates achievable with an internal substrate surface (6) of the nozzle (18) “polished” as above described. The plot line which contains Point T (71) and Point S (72) represents flow rates achievable with an internal substrate surface (6) of the nozzle (18) having a lubricious layer (7). The increased flow rate of the sheath fluid stream (10) achievable by coupling a lubricious layer (7) to the internal substrate surface of the nozzle (18) which terminates at the nozzle orifice (19) can allow substantial increases in the flow rate of the sheath fluid stream (9) in coaxial laminar relation to the sample fluid stream (10) which can allow for ratios of the sample fluid stream (10) to the sheath fluid stream (9) substantially lesser ratios than conventionally achievable. For example, utilizing a configuration of the nozzle assembly (43) which provides a nozzle (18) having a lubricious layer (7) (see FIG. 13) (and certain embodiments which further provide a sample fluid conduit having a lubricious layer (7) as shown in FIG. 16 the sample fluid stream (10) can join the sheath fluid stream (9) in coaxial laminar flow at a flow rate of about one volume per unit time, while the sheath fluid stream (9) can flow into coaxial laminar flow surrounding the sample fluid stream (10) at a rate of greater than 100 volume units per unit time, or as to certain embodiments adjusted between 100 volume units per unit time to 300 volume units per unit time. Corresponding increases in concentricity of each of the plurality of particles (17) with the longitudinal axis (67) of the fluid stream (8) can be achieved in this manner. Alternately, the ratio of the flow rate of the sample fluid stream (10) and the sheath fluid stream (9) can be established at conventional levels (for example 1:100) with the corresponding advantage of reduced pressure of the fluid stream (8) as compared to conventional pressures of the fluid stream (8) which are typically maintained at between about 40 psi and 60 psi, or increased functionality, viability or fertility of the sperm cells (26) analyzed or sex sorted. Alternately, as to certain embodiments of the invention which couple a lubricious layer (7) to at least a portion of the substrate surface which define the sample fluid flow path or the sheath fluid flow path the sheath fluid stream (9) and the sample fluid stream (10), the pressure of the sheath fluid stream (9) and the sample fluid stream (10) can be operated at pressures which are actually greater than conventional pressures such as 50 psi, 60 psi, 70 psi, 80 psi, 90 psi or 100 psi (an increase over convention pressures for flow sort of cells of: between about 5 psi and about 10 psi, between about 10 psi and about 15 psi, between about 15 psi and about 20 psi, between about 20 psi and about 25 psi, between 25 psi and about 30 psi, between about 30 psi and about 35 psi, between about 35 psi and about 40 psi, between about 40 psi and about 45 psi, and between about 45 psi and about 50 psi) with a resulting increase in fluid stream (8) velocity of between about 1 m/s about 5 m/s, between about 5 m/s and about 10 m/s, between about 10 m/s and about 15 m/s, between about 15 m/s and about 20 m/s, between about 20 m/s and about 25 m/s. The increases in sheath fluid and sample fluid pressure and fluid stream velocity can be achieved without a reduction (and as to certain embodiments of the invention an increase) in the viability of sperm cells analyzed and sorted.

Because the light beam (22) which interrogates the fluid stream (8) typically are aligned to intercept the fluid stream (8) substantially coincident with the longitudinal axis (67), increased centricity of each of the plurality of particles (17) or for a plurality of particles on average, allows increased consistency in the amount of light (23) generated between each of the plurality of particles (17) interrogated of like kind. As one non-limiting example, each of a plurality of Y-chromosome bearing sperm cells (41) which present a similar amount of bound fluorochrome to a laser beam should emit a substantially similar amount of light assuming the laser beam interrogates each sperm cell in substantially similar manner. Increasing centricity of each of the plurality of sperm cells (26) acts to correspondingly increase the likelihood that each of the sperm cells (26) will be interrogated in a similar manner.

Now referring specifically to FIG. 2, this can result in increased resolution between the population of X-chromosome bearing sperm cells (40) and Y-chromosome bearing sperm cells (41) as viewed in the corresponding histogram (34) either as increased peak to peak distance (77) or a shown by the corresponding bivariate plot (80) as an increased valley depth (74) between the peaks (78)(79). An increased resolution of two populations represented in a plurality of particles can allow for collection of a greater number each of the plurality of particles (17) per unit time or an increase in the purity of the two populations collected or both.

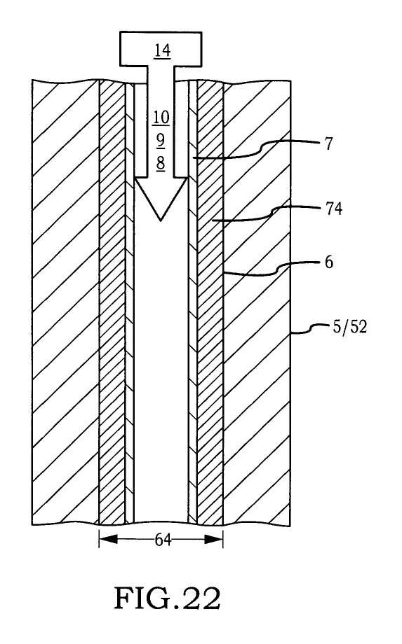

Now referring to FIG. 22, a further embodiment of the invention includes a sample fluid conduit (52) which provides a sheath element (74) engaged with the internal substrate surface (6) of the sample fluid conduit (52). For example, a sample fluid conduit (52) of stainless steel having a sample fluid conduit internal diameter (64) of about 180μ can be obtained from Popper & Sons, P.O. Box 128, New Hyde Park, N.Y., PN 9537. A sheath element (74) of extruded polyamide having an external diameter of about 140μ and an internal diameter of about 112μ can be obtained from Microlumen, 7624 Bold Cypress Place, Tampa, Fla., PN 044-I. Other dimensional forms of sheath element(s) (74) can also be obtained having for example an internal diameter of 221μ and an external diameter of 264μ which can be used as a sheath element for other dimensional forms of sample fluid conduits (52). The sheath element (74) can be slidely engaged in the sample fluid conduit (52) to produce an internal substrate surface (6) of greater lubricity than conventional extruded stainless steel sample fluid conduits (52) and providing a lesser diameter sample fluid conduit orifice (54). As to certain embodiments of the invention, the internal substrate surface (6) of the sheath element (74) can be treated with one or more of the above-described compositions to provide a lubricious layer (7). Other embodiments of the invention can utilize smaller diameter forms of the sheath element (74) which slidely engage larger diameter forms of the sheath element (74). The smaller diameter forms of the sheath element (74) being treated with compositions as above-described to provide a lubricious layer (7).

Various embodiments of the invention can provide a flow path having a lubricious layer (7) (whether in whole or in part) (also referred to as a lubricious flow path (3)) which can substantively alter flow characteristics of a flow of a fluid (11) in the context of analyzing a plurality of particles (17). The interdependency of the lubricious flow path (3) and such altered flow characteristics of the flow of fluid (11) makes achievable the enhanced performance characteristics of a lubricious microfluidic flow path system (1). In the context of a flow cytometer (2) embodiment of the invention, the speed of analysis of a plurality of particles (17) and the separation of a plurality of particles (17) (including embodiments of the invention configured to analyze and sort sperm cells (26)), the yield of analytical data and collection of separated cells (including but not limited to sperm cells (26)), and the purity of the populations of separated cells (including but not limited to X-chromosome bearing and Y-chromosome bearing populations of sperm cells (26) can each separately, or two in various combinations, or all be increased relative to a conventional flow cytometer device (including those flow cytometers (2) configured to analyze and sex sort sperm cells (26).

### Example 1

Modification of Flow Cytometer Nozzle Assemblies and Operation of Flow Cytometers. Four substantially identical conventional nozzle assemblies (43) for a MOFLO® SX flow cytometer manufactured by Beckman Coulter, Fort Collins, Colo. were utilized to provide the standard and experimental nozzle assemblies for comparative trials. A lubricious layer (7) of the hydrophilic polymer poly(vinyl pyrollidone) was applied to the internal substrate surface (6) of the sample fluid conduit (52) and the internal surface of the nozzle (18) of two of the four nozzle assemblies (43). All four of the nozzle assemblies provided a 70 μM diameter nozzle orifice (19). Each of the two standard nozzle assemblies (43) and each of the two experimental nozzle assemblies (43) having the lubricious layer (7) applied as above described were installed onto otherwise substantially identical MOFLO SX® flow cytometers and operated side by side.

### Composition of Solutions.

Sperm Staining Solution: 3 grams/L BSA, 80 mM HEPES, 1 mM MgCl, 40 mM NaCl, 5 mM KCl, 10 mM NaHCO3, 5 mM NaCH3COCOO, 20 mM Glucose, 25 mM NaCH3CHOHCOO, adjusted to pH 7.2 with NaOH in polished water.
Hoechst 33342 Working Solution: 8.9 mM of (2-(4-Ethoxyphenyl)-5-(4-methyl-1-poperazinyl)-2-5-bis-1H-benzimidazole in polished water.
Sample Holding Solution: 4% egg yolk (vol/vol), 3 grams/L BSA, 80 mM HEPES, 1 mM MgCl, 40 mM NaCl, 5 mM KCl, 10 mM NaHCO3, 5 mM NaCH3COCOO, 20 mM Glucose, 25 mM NaCH3CHOHCOO, 250 mg/L FD&C Red40, adjusted to pH 6.0 with HCl in polished water.
Sheath Fluid: 130 mM TRISma Base, 45 mM Citric Acid, 120 mM Fructose, adjusted to pH 6.8 with HCl in polished water.
Sperm Catch Fluid: 20% egg yolk (vol/vol), 130 mM TRISma Base, 45 mM Citric Acid, 120 mM Fructose, adjusted to pH 6.8 with HCl in polished water.
Glycerolizing Solution: 135 mM glycerol, 130 mM TRISma Base, 45 mM Citric Acid, 120 mM Fructose, adjusted to pH 6.8 with HCl in polished water.
Freezing Solution: 20% egg yolk (vol/vol), 65 mM glycerol, 130 mM TRISma Base, 45 mM Citric Acid, 120 mM Fructose, adjusted to pH 6.8 with HCl in polished water.
Preparation of Sperm Cells for Sorting. Freshly Ejaculated Semen was Collected from proven bulls with an artificial vagina and kept in incubation cabinet at 24-27° C. for up to 8 hours prior to taking samples for staining. Sperm cells (26) can be suspended at 1.6×10⁸ sperm cells (26) per mL in Staining Solution with 1 microliter of Hoechst 33342 Working Solution (capitalized terms refer to the above defined composition of solutions) added per 1.0×10⁷ sperm cells (26) and incubated at 35-38° C. for 45-60 minutes, then mixed with an equal volume of Sample Holding Solution, filtered through a 50 micron pore CellTrics Filter (Partec, GmbH, Germany) and held for up to 2 hours as sorting sample.
Alignment of Instrumentation Analysis: A MoFlo SX or MoFlo XDP flow cytometer (Beckman Coulter, Miami, Fla. USA), equipped with MoFlo SX sperm sorting nozzle (XY, Inc. Fort Collins, Colo., USA) and a Vanguard 350-HMD 355 laser with 125-150 mW beam energy, can be operated with Sheath Fluid at a standard pressure of 40 psi, or at an experimental pressure, as described in examples. Stained sperm cells (26) can be used to align and orient particle flow to maximize resolution between live and dead sperm cell populations.
Establishment of Drop Delay Calibration. 100 microliters of stained sperm cells (26) in Sample Holding Solution are added to 900 microliters Sheath Fluid and placed in sample tube. The resulting sample fluid (10) containing stained sperm cells (26) can be pressurized to achieve a particle analysis rate of about 1000 particles per second. Particles appearing as live sperm on the flow cytometer histogram are gated for collection and 200 particles are collected onto a microscope slide. This procedure is repeated at a multiple of drop delay calibration settings to determine the optimal drop delay setting able to maximize the number of sperm in one collection.
Bulk Sorting of Live Sperm: Stained sperm cells (26) diluted in Sample Holding Solution can be placed in the particle source and pressurized at a pressure sufficient to deliver 40,000 sperm cells (26) per second to through the nozzle orifice of the flow cytometer. Live X-chromosome bearing sperm cells (40) and Y-chromosome bearing sperm cells (410) were gated for collection at rates between 8,000 and 14,000 live sperm cells per second and collected into 7 mL of Sperm Catch Fluid until 33 mL volume was collected (total volume 40 mL). Depending on conditions used, this corresponds to between 25 and 100 live million sperm cells (26) collected. Sorted sperm cells (26) were cooled from about 22° C. to about 4° C. gradually over 45 minutes and held for about an additional 45 minutes at 4° C. 20 mL of Glycerolization Solution was added and mixed and sample was held for a about 15 minutes at 4° C. with this step repeated to add a second amount of 20 ml of Glycerolization Solution.
Cryopreservation of Sperm: Sorted sperm cells (40)(41) were centrifuged at 800×g for 20 minutes. Supernatant was removed and the remaining sperm cell pellet was resuspended in 5 mL of Freezing Solution. About 220 μL of suspended sperm cells (26) can be deposited into each 0.25 cc TBS cryopreservation straw (IMV Technologies Group, L′Aigle, France). Straws can be rapid cooled over nitrogen vapor at a rate of about 5° C. per minute to a point of about minus 110° C. and plunged into liquid nitrogen.
Analysis of Sperm Quality: Straws containing sorted sperm cells (40)(41) are thawed by removing from liquid nitrogen and immediately plunging into a 39° C. water batch for 30 seconds. Sperm cells are removed from cryopreservation straws into 5 ml polypropylene tubes and held at 36° C. and immediately inspected under at microscope slide coverslip on top of a microscope slide kept at about 35° C. on a warmed stage. Visual motilities are expressed as a percentage of visually motile sperm of 100 sperm cells counted. Intact acrosomes are expressed as percentage of visually intact acrosomes (the acrosome is not intact if fully or partially displaced or lacking) of 100 sperm counted. 2 or 3 replicate straws are counted and their average is used.
Experimental Design. A first of the two MOFLO SX® flow cytometers (2) installed with a standard nozzle assembly (43) was operated at the standard conditions with the Sheath Fluid pressurized at 40 psi to generate a fluid stream (8) exiting the nozzle orifice (19) having a stream velocity of about 23.40 meters per second and a drop drive frequency of about 63.10 KHz applied to the nozzle (18) to generate droplets (20) having drop volume of about 1.43 nL and a drop radius of about 69.84 μM. A second of the two MOFLO SX® flow cytometers installed with a standard nozzle assembly (43) was operated at the experimental conditions with the sheath fluid pressurized at 40 psi, 50 psi, 70 psi, or 85 psi with the drop drive frequency adjusted in the range of 63.10 KHz and 119.00 KHz applied to the nozzle (18) to generate droplets having a drop radius in the range of 62.13 μM and 69.84 μM and corresponding drop volume in the range of 1.00 nL and 1.43 nL. The first and second MOFLO SX® flow cytometers installed with an experimental nozzle assembly (43) were operated at the experimental conditions with the sheath fluid pressurized at 40 psi, 50 psi, 70 psi, or 85 psi with the drop drive frequency adjusted in the range of 63.10 KHz and 119.00 KHz applied to the nozzle (18) to generate droplets having a drop radius in the range of 62.13 μM and 69.84 μM and corresponding drop volume in the range of 1.00 nL and 1.43 nL. As to each experimental condition a minimum of two different samples of labeled sperm cells (26) were analyzed and sex sorted as above described.
Minimum Drop Radius. Fluid stream (8) pressure was fixed at 70 psi to generate a fluid stream (8) velocity of about 30.96 meters per second the drop drive frequency can be adjusted in a range of about 83.50 KHz about 120 KHz to generate droplets (20) having a corresponding drop radius in the range of about 69.84 μM to about 62.13 μM as shown by Table 3. Sperm cells were stained, sex sorted, frozen and thawed, as above described. Zero hour post thaw motility and three hour acrosomal integrity were assessed for each drop radius. Sperm cells (26) sex sorted in droplets (20) having a drop radius of less than 64.0 μM can have a lower motility and a lower acrosomal integrity than for sperm cells (26) sex sorted in droplets having a minimum drop radius of greater than 64.0 μM. Experimental conditions were established to preclude experimental conditions as to sheath fluid (9) pressure, fluid stream (8) velocity, and drop drive frequency which resulted in drop radius of less than 64.0 μM.
Results. Each standard condition and experimental condition with respect to sheath pressure over the range of 40 psi and 85 psi generated the expected corresponding increase in stream velocity, as shown in Table 3. For the experimental condition (referred to in Table 4 as LUB followed by the experimental pressure) at LUB 70 psi in which the droplet radius was less that 64 μM a decrease in motility and decrease in acrosomal integrity of the sorted sperm cells (26) was observed as compared to the standard operating condition (referred to as STD followed by the pressure). For the experimental conditions LUB 40, LUB 50, LUB 85 as compared to corresponding the standard conditions (STD) in which the droplet radius was above 64 μM an increase in motility and an increase in acrosomal integrity of the sorted sperm cells (26) was observed.

TABLE 3

| | | Drop Drive | | | % | |
|---|---|---|---|---|---|---|
| | Stream | Frequency | Drop | Drop | change | % |
| | Velocity | (DDF) | Volume | Radius | in Drop | change |
| PSI | (meters/sec) | (KHz) | (nanoL) | (μM) | Radius | in DDF |
| 40 | 23.40 | 63.10 | 1.43 | 69.84 | 100.0% | 100% |
| 50 | 26.16 | 80.00 | 1.26 | 67.11 | 96.1% | 127% |
| 70 | 30.96 | 119.00 | 1.00 | 62.13 | 89.0% | 189% |
| 85 | 34.11 | 117.00 | 1.12 | 64.52 | 92.4% | 185% |

TABLE 4

| | 0 Hour Post-Thaw Motility | 3 Hour Intact Acrosome |
|---|---|---|
| LUB 40/STD 40 | 100.65% | 100.04% |
| LUB 50/STD 40 | 104.46% | 120.71% |
| LUB 50/STD 50 | 95.72% | 105.41% |
| LUB 85/STD 40 | 112.03% | 108.49% |
| LUB 70/STD 40 | 88.34% | 94.27% |
| LUB 70/STD 70 | 92.18% | 97.17% |

While the working example above describes particular: flow cytometers, lubricious materials and lubricious flow paths, compositions of solutions, species of sperm cells from bovine bulls, methods of staining sperm cells, methods of analysis and flow sorting of sperm cells, methods of cryopreserving sperm cells, methods of analysis of sperm cell quality, and the like; the invention is not so limited, and the working examples along with the description and figures provided are intended to be sufficient to allow a person of ordinary skill in the art to make and use a numerous and wide variety of embodiments of the invention including the best mode which may be applied generally to cells or sperm cells obtained from a wide variety of animals or male mammals or cell lines processed using various methods of staining or other methods which allow assessment of particle characteristics (such as the amount of DNA within the nucleus of sperm cells) or otherwise allow identification of subpopulations of cells (such as Y-chromosome bearing sperm cells from X-chromosome bearing sperm cells) by flow cytometry or other microfluidic devices in which cells or sperm cells are transferred within conduits under pressure for the purpose of increasing the rate at which the cells or sperm cells can be analyzed or sorted while maintaining or increasing the cell function or sperm cell viability.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of an “oscillator” should be understood to encompass disclosure of the act of “oscillating”—whether explicitly discussed or not—and, conversely, were there effectively disclosure of the act of “oscillating”, such a disclosure should be understood to encompass disclosure of an “oscillator” and even a “means for oscillating.” Such alternative terms for each element or step are to be understood to be explicitly included in the description.

In addition, as to each term used it should be understood that unless its utilization in this application is inconsistent with such interpretation, common dictionary definitions should be understood to included in the description for each term as contained in the Random House Webster's Unabridged Dictionary, second edition, each definition hereby incorporated by reference.

Thus, the applicant(s) should be understood to claim at least: i) each of the lubricious microfluidic flow path devices herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Moreover, the claims set forth below are intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

We claim:

1-71. (canceled)

72. A flow cytometer, comprising:
a) a nozzle having a nozzle orifice;
b) a sample fluid conduit having an internal substrate surface configured to define a sample fluid flow path which terminates in a sample injection orifice a distance from said nozzle orifice, and wherein an external substrate surface of said sample fluid conduit and an internal substrate surface of said nozzle define a sheath fluid flow path;
c) a lubricious layer coupled to at least a portion of said substrate surface which defines said sample fluid flow path or said sheath fluid flow path.

73. A flow cytometer as described in claim 72, further comprising:
a sample fluid which flows in said sample fluid flow path; and
a sheath fluid which flows within said sheath fluid flow path, wherein said sample fluid exits said sample injection orifice to flow within said nozzle surrounded by said sheath fluid to exit said nozzle orifice as a fluid stream.

74. A flow cytometer as described in claim 73, further comprising an oscillator which oscillates said nozzle to form droplets which break away from said fluid stream a distance below said nozzle orifice.

75. A flow cytometer as described in claim 74, wherein said lubricious layer coats a sufficient portion of said substrate surface which defines said sample fluid flow path or said sheath fluid path to increase velocity of said fluid stream from which droplets break away at a distance below said nozzle orifice.

76. A flow cytometer as described in claim 74, wherein each of said sheath fluid stream and said sample fluid stream have a pressure adjusted within a range of about 20 psi and 100 psi, and wherein without increase of said pressure said lubricious layer coats a sufficient amount of said substrate surface to allow an increase in velocity of said fluid stream from which droplets break away at said distance below said nozzle orifice.

77. A flow cytometer as described in claim 76, wherein said oscillator has a drop drive frequency adjustable to increase rate of droplets formed in said fluid stream having said increase in velocity.

78. A flow cytometer as described in claim 74, further comprising a plurality of particles entrained in said sample fluid which flows surrounded by said sheath fluid, and wherein each one of said droplets which break away from said fluid stream beneath said nozzle orifice contains at least one of said plurality of particles.

79. A flow cytometer as described in claim 78, wherein each of said sheath fluid and said sample fluid have a pressure adjusted within a range of about 20 psi and 100 psi, and wherein said lubricious layer coats a sufficient amount of said substrate surface to allow an increase in said pressure of said sheath fluid or said sheath fluid without reduction in viability of said plurality of particles contained in said droplets.

80. A flow cytometer as described in claim 78, wherein said lubricious layer coats a sufficient amount of said substrate surface to allow an increase in viability of said plurality of particles contained in said droplets.

81. A flow cytometer as described in claim 74, wherein said sample fluid which exits said sample injection orifice to flow within said nozzle surrounded by said sheath fluid has a reduced cross section dimension selected from the group consisting of: between about 0.0080 inches and about 0.0075 inches, between about 0.0075 inches and about 0.0070 inches, between about 0.0070 inches and about 0.0060 inches, between about 0.0060 inches and about 0.0050 inches.

82. A flow cytometer as described in claim 81, wherein said nozzle has a nozzle orifice having a internal dimension selected from the group consisting of: between about 70 μm and about 65 μm, between about 65 μm and about 60 μm, between about 60 μm and about 55 μm, between about 55 μm and about 50 μm.

83. A flow cytometer as described in claim 72 wherein said plurality of plurality of particles comprises a plurality of cells.

84. A flow cytometer as described claim 72 wherein said plurality of sperm cells is selected from the group consisting of: bovine sperm cells, ovine sperm cells, equine sperm cells, swine sperm cells, cervine sperm cells, feline sperm cells, canine sperm cells, rodent sperm cells, cetacean sperm cells, rabbit sperm cells, primate sperm cells.

85. A flow cytometer as described in claim 72, wherein said lubricious layer comprises one layer of lubricious material capable of binding to said substrate surface.

86. A flow cytometer as described in claim 72, wherein said lubricious layer comprises a first binding layer capable of binding to said substrate surface which affords a plurality of reactive groups and a bound layer which reacts with said reactive groups of said binding layer.

87. A flow cytometer as described in claim 86, wherein said binding layer has a plurality of hydroxyl groups and said bound layer comprises a polyvinyl pyrrolidone.

88. A method of flow cytometry, comprising:
a) providing a nozzle having a nozzle orifice;
b) providing sample fluid conduit having an internal substrate surface configured to define a sample fluid flow path which terminates in a sample injection orifice a distance from said nozzle orifice, and wherein an external substrate surface of said sample fluid conduit and an internal substrate surface of said nozzle define a sheath fluid flow path; and
c) coupling a lubricious layer to at least a portion of said substrate surface which defines said sample fluid flow path or said sheath fluid flow path.

89. The method of flow cytometry as described in claim 88, further comprising the steps of:
a) ejecting said sample fluid from said sample injection orifice;
b) surrounding said sample fluid ejected from sample injection orifice with said sheath fluid in said nozzle; and
c) passing said sample fluid surrounded by said sheath fluid through said to generate a fluid stream below said nozzle.

90. The method of flow cytometry as described in claim 89, further comprising the step of oscillating said nozzle to form droplets in said fluid stream which break away a distance below said nozzle orifice.

91. The method of flow cytometry as described in claim 90, further comprising the step of coating a sufficient portion of said substrate surface to allow an increase in viability of said plurality of particles contained in said droplets.

92. The method of flow cytometry as described in claim 91, further comprising the steps of:
a) adjusting said pressure of said sheath fluid stream and said sample fluid stream within a range of about 20 psi and 100 psi; and
b) coating a sufficient portion of said substrate surface to allow an increase in said pressure of said sheath fluid or said sheath fluid with an increase in viability of said plurality of particles contained in said droplets.

93. The method of flow cytometry as described in claim 92, wherein said increase in said pressure of said sheath fluid or said sample fluid without reduction in viability of said plurality of particles contained in said droplets is selected from the group consisting of: between about 5 psi and about 10 psi, between about 10 psi and about 15 psi, between about 15 psi and about 20 psi, between about 20 psi and about 25 psi, between 25 psi and about 30 psi, between about 30 psi and about 35 psi, between about 35 psi and about 40 psi, between about 40 psi and about 45 psi, and between about 45 psi and about 50 psi.

94. The method of flow cytometry as described in claim 93, further comprising the step of increasing said velocity of said fluid stream by increase in said pressure of said sheath fluid or said sample fluid without reduction in viability of said plurality of particles contained in said droplets.

95. The method of flow cytometry as described in claim 94, wherein said increase in said velocity of said fluid stream is selected from the group consisting of: between about 1 m/s about 5 m/s, between about 5 m/s and about 10 m/s, between about 10 m/s and about 15 m/s, between about 15 m/s and about 20 m/s, between about 20 m/s and about 25 m/s.

96. The method of flow cytometry as described in claim 90, further comprising the step of coating a sufficient portion of an internal substrate surface of said a sample fluid conduit with said lubricious layer to allow use of a reduced cross section dimension of said sample fluid flow path without reduction in viability of said plurality of particles contained in said droplets.

97. The method of flow cytometry as described in claim 96, wherein said cross section dimension is selected from the group consisting of: between about 0.0080 inches and about 0.0075 inches, between about 0.0075 inches and about 0.0070 inches, between about 0.0070 inches and about 0.0060 inches, between about 0.0060 inches and about 0.0050 inches.

98. The method of flow cytometry as described in claim 88, wherein said plurality of particles comprises a plurality of cells.

99. The method of flow cytometry as described in claim 88, wherein said plurality of sperm cells is selected from the group consisting of: bovine sperm cells, ovine sperm cells, equine sperm cells, swine sperm cells, cervine sperm cells, feline sperm cells, canine sperm cells, rodent sperm cells, cetacean sperm cells, rabbit sperm cells, primate sperm cells.

100. The method of flow cytometry as described in claim 88, wherein said step of coupling a lubricious layer to at least a portion of said substrate surface which defines said sample fluid flow path or said sheath fluid flow path comprises the step of coupling one layer of lubricious material capable of binding to said substrate surface.

101. The method of flow cytometry as described in claim 100, wherein said step of coupling a lubricious layer to at least a portion of said substrate surface which defines said sample fluid flow path or said sheath fluid flow path comprises the steps of:
a) coupling a first binding layer capable of binding to said substrate surface which affords a plurality of reactive groups; and
b) reacting a bound layer with said reactive groups of said binding layer.

102. The method of flow cytometry as described in claim 101, wherein said binding layer has a plurality of hydroxyl groups and said bound layer comprises a polyvinyl pyrrolidone.
